(19) United States
(12) Patent Application Publication
(10) Pub. No.: US 2025/0340824 A1
(43) Pub. Date: Nov. 6, 2025

(54) PROCESSES AND SYSTEMS OF CULTURING ALGAE AND MIXING GROWTH MEDIUM IN AN ALGAL AQUACULTURE POND

(71) Applicant: Neste Oyj, Espoo (FI)

(72) Inventors: Jeffrey Scott KANEL, Porvoo (FI); Cecil Calvert CHURN, III, Porvoo (FI); David Robert BRYANT, Porvoo (FI); Cecilia Alexandra STILL, Porvoo (FI)

(73) Assignee: Neste Oyj, Espoo (FI)

(21) Appl. No.: 18/718,649

(22) PCT Filed: Dec. 30, 2022
(86) PCT No.: PCT/US2022/054327

§ 371 Date: Jun. 11, 2024

Related U.S. Application Data
(60) Provisional application No. 63/295,559, filed on Dec. 31, 2021.

(51) Int. Cl. C12N 1/12; A01G 33/00; C12M 1/00; C12M 1/02; C12M 1/34

(52) U.S. Cl. CPC C12N 1/12; A01G 33/00; C12M 23/18; C12M 27/00; C12M 41/32; C12M 41/36

(57) ABSTRACT

There are disclosed processes and systems of culturing and mixing algal growth medium in at least one open algal aquaculture pond for a continuous flow system without requiring mechanical mixing devices. The process involves constructing an open algal aquaculture pond possessing a fetch selected as a function of a specified wind speed and a wave mixed layer (WML) defined by a specified depth from the surface of the pond; supplying algal growth medium to the open algal aquaculture pond, the pond having at least partially a manmade configuration; and culturing algae in the algal growth medium in the open algal aquaculture pond; and wherein a ratio of the WML of the algal growth medium to a total depth of the algal growth medium in the pond is greater than about 0.2. Also disclosed are uses of the mixed algal aquaculture medium and systems for culturing algae.

### CROSS-REFERENCE TO RELATED APPLICATION

This application incorporates the entire contents of U.S. Provisional Patent Application No. 63/295,559 filed on Dec. 31, 2021.

### FIELD

The present disclosure is directed to processes and systems of culturing algae and mixing algal growth medium in at least one open algal aquaculture pond possessing a fetch to mix the algal growth medium. Also disclosed are algal culturing systems for mixing an algal growth medium, a mixed algal aquaculture medium, and the use of the mixed algal aquaculture pond and systems for cultivating algae.

### BACKGROUND INFORMATION

There is increasing interest in using algal biomass as a key intermediate for a plethora of sustainable products, such as a source of renewable energy, as a mode to safely and efficiently capture carbon dioxide from the atmosphere for carbon sequestration, and as a renewable source of chemical intermediates. The grown algae are typically harvested to form an algal concentrate.

The cost of culturing algae is significant, and there is a need to reduce the associated capital and operating costs in order to make the overall process of converting algal biomass into valuable components more economically attractive. Thus, one of the key challenges of handling algal aquaculture is the need to minimize both capital and operating costs. The art teaches that there is a need to mechanically agitate algal aquaculture ponds in order to adequately mix nutrients and minerals that are essential for suitable algal growth in the algal aquaculture ponds, such mechanical agitation occurring both in a water column and in the bulk of the algal aquaculture pond.

There are many existing designs using mechanical agitation, such as paddle wheels U.S. Pat. No. 9,295,206 B2 and liquid jets, to keep the algal aquaculture mixed. Several companies utilize raceway ponds including Nature Beta Technologies located in Eilat, Israel and Cyanotech, located in Hawaii. Additionally, U.S. Pat. No. 4,958,460 discloses a process for the reproduction and harvesting of algae of the genus Dunaliella and associated bacteria, with a specific cross-sectional width to depth ratio of the algal aquaculture pond to harvest the algae. Further, U.S. Pat. No. 10,524,432 discloses a bioreactor system for controlling the cultivation of an aquatic plant culture and algal aquaculture ponds having a horizontal raceway design.

At large scale, existing raceway-style algal aquaculture ponds are very expensive to construct such that they are suitable for culturing algae, they use high-cost liners, paddlewheel agitators, and process control equipment. Furthermore, during scale up, the art teaches the use of multiple algal aquaculture ponds with mechanical mixers such as paddlewheels. Such algal aquaculture ponds are scaled-up, for example, by keeping the size of the individual algal aquaculture ponds with paddlewheels constant, but increasing the number of these algal aquaculture ponds. For economical deployment of algal aquaculture ponds at large-scale for the production of biofuels, more robust and lower capital cost equipment is needed.

### SUMMARY

Described herein is a process of culturing algae and mixing algal growth medium in at least one open algal aquaculture pond for a continuous flow system, the process comprising: (a) constructing the open algal aquaculture pond, the algal aquaculture pond possessing a fetch selected as a function of a specified wind speed and a wave mixed layer (WML) defined by a specified depth from the surface of the algal aquaculture pond; (b) supplying algal growth medium to the open algal aquaculture pond, the open algal aquaculture pond having at least partially a manmade configuration and being in communication with a harvester for separating algal biomass from the algal growth medium; and (c) culturing algae in the algal growth medium in the open algal aquaculture pond by mixing at least part of the algal growth medium and/or circulating the algal growth medium within the algal aquaculture pond, the mixing being a function of the wind speed and the fetch; and wherein a ratio of the WML of the algal growth medium to a total depth of the algal growth medium in the algal aquaculture pond is greater than about 0.2.

Also disclosed is a continuous flow algal culturing system for mixing an algal growth medium, the system comprising: at least one or more of an open algal aquaculture pond, the open algal aquaculture pond possessing a fetch selected as a function of a specified wind speed and a wave mixed layer (WML) defined by a specified depth from the surface of the algal aquaculture pond; at least a part of the open algal aquaculture pond having at least partially a manmade configuration and being in communication with a harvester for separating algal biomass from the algal growth medium; and wherein a ratio of the WML of the algal growth medium to a total depth of the algal growth medium in the algal aquaculture pond is greater than about 0.2.

There is further provided a mixed algal aquaculture medium obtainable by a process disclosed herein.

The use of a mixed algal aquaculture medium as disclosed herein, or the system as disclosed herein, for cultivating or culturing algae is also provided.

### BRIEF DESCRIPTION OF THE DRAWINGS

Some of the features and advantages of the disclosure have been stated. Other advantages will become apparent as the disclosure proceeds, taken in conjunction with the accompanying drawings, in which:

FIG. 1 illustrates an exemplary embodiment of an open algal aquaculture pond 1 for a continuous flow system, wherein the algal aquaculture pond 1 possesses a fetch 3 (measured in meters or “m”) selected as a function of a specified wind speed 5 (measured in meters/second or “m/s”) and a wave mixed layer 7 (WML, measured in “meters” or “m”) defined by a specified depth of the total depth of the pond (D) 9 (measured in meters or “m”) from the surface of the algal aquaculture pond 1.

FIG. 2 illustrates an exemplary embodiment of an open algal aquaculture pond 1 or a system in accordance with an aspect of the present disclosure.

FIG. 3 illustrates an exemplary soil classification methodology for use in building an open algal aquaculture pond 1 in accordance with the present disclosure.

FIG. 4 illustrates an exemplary wind rose plot to assist in orienting an open algal aquaculture pond 1 in accordance with an aspect of the present disclosure.

FIG. 5 illustrates an exemplary system with a plurality of open algal aquaculture ponds 1 (each is depicted as an “algal growth pond”) in accordance with an aspect of the present disclosure in parallel.

FIG. 6 illustrates an exemplary system with a plurality of open algal aquaculture ponds 1 (each is depicted as an “algal growth pond”) in accordance with an aspect of the present disclosure in series.

### DETAILED DESCRIPTION

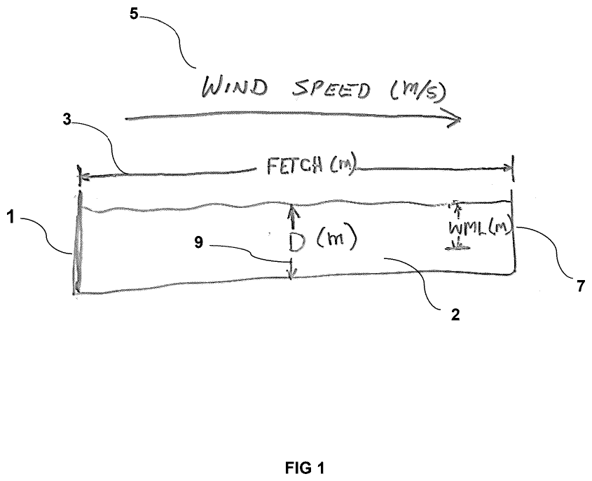

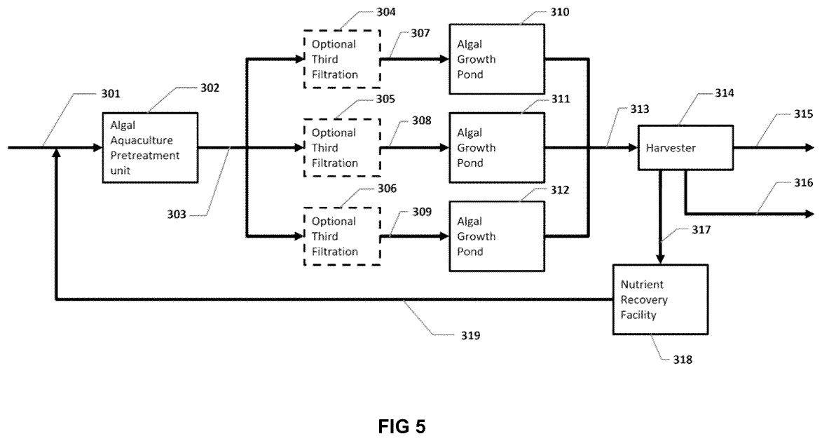

In an exemplary embodiment of the present disclosure there is provided a process of culturing algae and mixing algal growth medium 2 in at least one open algal aquaculture pond 1 for a continuous flow system, such as that shown in FIG. 1, the process comprising: (a) constructing the open algal aquaculture pond 1, the algal aquaculture pond 1 possessing a fetch 3 selected as a function of a specified wind speed 5 and a wave mixed layer (WML) 7 defined by a specified depth from the surface of the algal aquaculture pond 1; (b) supplying algal growth medium 2 to the open algal aquaculture pond 1, the open algal aquaculture pond 1 having at least partially a manmade configuration and being in communication with a harvester (e.g. 314, FIG. 5) for separating algal biomass from the algal growth medium; and (c) culturing algae in the algal growth medium 2 in the open algal aquaculture pond 1 by mixing at least part of the algal growth medium 2 and/or circulating the algal growth medium 2 within the algal aquaculture pond 1, the mixing being a function of the wind speed 5 and the fetch 3; and wherein a ratio of the WML 7 of the algal growth medium 2 to a total depth 9 of the algal growth medium in the algal aquaculture pond is greater than about 0.2.

As used herein, the term “algal growth medium” is understood to refer to algal growth medium 2 existing in the algal aquaculture pond 1, as well as any additional medium such as aqueous medium which is added to the algal aquaculture pond to supplement the algal growth medium already in the algal aquaculture pond and thus to form the algal growth medium. In an embodiment, the algal growth medium comprises water and one or more of mineral salts, heavy metals, algae, microalgae, algal predators, algal competitors, or (residual) nutrients.

The algal growth medium 2 may be derived from any suitable source, including, but not limited to, an ocean, a sea, a lake, a river or stream, an underground aquifer, a canal, an irrigation canal, a wastewater discharge, effluent from an aquaculture facility, such as one that raises shrimp, fish, crustaceans, mollusks, shellfishes or combinations thereof.

In certain embodiments, the algal growth medium 2 comprises additional aqueous medium (“make up” medium) that is periodically or continuously provided to the algal aquaculture pond 1 to supplement the algal growth medium existing in the algal aquaculture pond 1. If the algal growth medium 2 in the algal aquaculture pond 1 comprises mineral salt, it is critical that the salinity of the additional aqueous medium fed to the algal aquaculture pond 1 is less than the highest salinity experienced by the algae in the algal growth/algal aquaculture pond 1. This is because water in the additional aqueous medium is replacing water that evaporates from the algal growth algal aquaculture pond and/or it is being used to increase the liquid depth of the algal aquaculture pond.

“Salinity” is a term that defines the total amount of dissolved inorganic solids (salts) in an aqueous solution. The typical salts found in natural waters may include sodium chloride, calcium and magnesium sulfates, bicarbonates, and carbonates. It is a standard practice to express salinity as parts per thousand (%), which is not a true percent but an approximation of the milligrams of salt per gram of water. In more general terms, salinity is indicated by the water source, such as a freshwater, a brackish water, a saline water, and a brine. Ranges of salinity are associated with these general terms and these ranges are defined as <0.5% (<0.05%) for freshwater, 0.5-30% (0.05-3%) for brackish water, 30-50% (3-5%) for saline water, and >50% (>5%) for a brine.

It should be noted that salts, especially sodium salts, combine with clays in the soil causing them to swell. This, in turn, tends to clog the pores, reducing the hydraulic conductivity. Thus, the hydraulic conductivity of the soils of open algal aquaculture ponds is anticipated to be very low when high salinity brines are used for the algal growth medium.

When the algal growth medium's 2 salinity is equal to or greater than that of seawater, then the algal aquaculture pond is preferably located near the ocean, sea, or other source of saline water, so that valuable fresh water is not required for the algal aquaculture pond. When located near the ocean or sea, there will typically be an inlet canal allowing ocean or seawater to feed an influent pump station or a tidal pool area that will serve as the feed for an influent pump station. Indeed, in one embodiment of the disclosure the system comprises an inlet canal allowing water such as ocean or seawater to enter the system, an influent pump station and/or a tidal pool area.

The algal growth medium 2 may comprise any components that are effective to promote the growth of algae. Many elements, including heavy metals, are required in trace amounts for optimum algal growth. Copper, for example, is necessary for the production of plastocyanin, a protein involved in electron transport in photosynthesis. Most algae exhibit some degree of inhibition to heavy metals depending on the algal type, concentration of the metal, the pH, and the concentration of chelators (naturally occurring or supplemented). The pH is important as it determines how much of the metal is present as a free ion, which is typically the more toxic form. Chelators of metals are interesting as they can prevent toxicity by making some metals non-bioavailable, while others, for example iron, can be made more bioavailable with chelation.

In certain embodiments, the algal growth medium 2 may be derived from lakes, rivers, streams, or combinations thereof. The lakes may comprise fresh water or saline water, such as that typically found in terminal lakes such as the Great Salt Lake in Utah. The rivers and streams may comprise different levels of mineral salt, heavy metals, algal predators, and algal competitors. It is preferable that the free cupric ion level in algal growth medium derived from lakes, rivers, and/or streams be as low as possible, and preferably less than twice that found in ocean water.

In certain embodiments, the algal growth medium 2 may be derived from underground aquifers, provided that the chemical composition of the algal growth medium is conducive to algal growth. Preferably, aqueous medium derived from underground aquifers have a free cupric ion level less than twice that in seawater, since copper is a known algaecide. When the algal growth medium is derived from an underground aquifer, it is also preferable that the divalent ion concentration be sufficiently low so that the algal growth rate is not negatively impacted by the presence of divalent ions including, but not limited to magnesium, calcium, and combinations thereof. Algae also tend to bioaccumulate heavy metals, such as arsenic that can substitute for phosphorus. Thus, it is preferred that the concentration of arsenic and other heavy metals are less than twice that found in seawater. Open ocean water typically has an arsenic concentration of 1-2 micrograms L⁻¹, while freshwater sources can be up to 10 micrograms L⁻¹ which is EPA's water quality standard limit for drinking water.

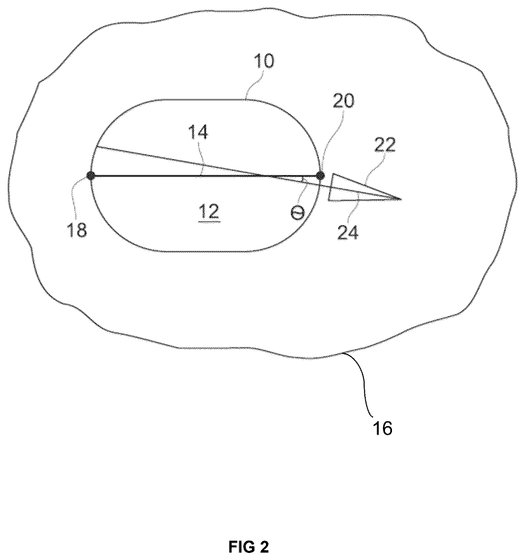

To provide more detail on the construction of an algal aquaculture pond 1 (or 10, FIG. 2) for use in the present disclosure, the algal aquaculture pond 1, 10 may be an open algal aquaculture pond. The open algal aquaculture pond can be applicable for almost all soils if a liner is used, but the expense of a liner makes such a system, in most cases, cost-prohibitive. Therefore, an important site selection criterion is the geological characteristics of the soil. A preferred soil is one that will hold water with minimal to no leakage.

In certain exemplary embodiments, the algal aquaculture pond 1, 10 is at least partially a manmade algal aquaculture pond, meaning that the algal aquaculture pond 1 is not naturally occurring in certain embodiments. For example, the pond 1, 10 has at least a partial purpose-built or synthetic structure in its “at least partially a manmade configuration”. In a further aspect, the pond is in communication with a harvester.

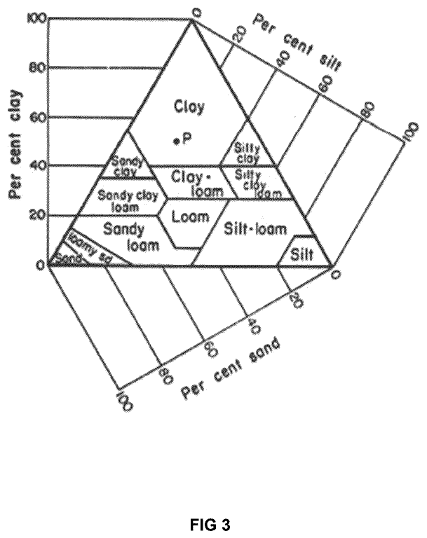

FIG. 3 represents a soil classification triangle that classifies soil types as a function of its silt, clay, and sand content. A soil that will hold water will have more clay content than sand or silt (see area labeled clay). Such soils can be found on the coastal plain of many areas around the world. These areas may be arable or non-arable with preference given to the later condition. A measure of the ease of both vertical and horizontal water movement in soil is referred to as hydraulic conductivity, K, and this can be used to define the range of soil types suitable for an unlined open algal aquaculture pond system. Preference is given to those soils with a hydraulic conductivity in the range of 10⁻⁶ to 10⁻¹⁰ cm sec⁻¹. The preference is to have the lowest possible hydraulic conductivity because soil conditions are rarely uniform. Thus, it is most preferable to have a hydraulic conductivity as low as possible, most preferably from 10⁻⁹ to 10⁻¹⁰ cm sec⁻¹.

The elevation of the groundwater can also be important for holding suitable medium in an algal growth/algal aquaculture pond 1, and it is preferable to have the groundwater level close to the elevation of the bottom of the algal aquaculture pond in order to reduce the hydraulic conductivity. Specifically, when operating in coastal environments, it is preferable to have the mean sea level to be within three meters of the elevation of the bottom of the algal aquaculture pond(s). It is even more preferable to have the groundwater level within one meter of the bottom of the algal aquaculture pond(s).

Open algal aquaculture ponds (e.g. 1, 10) are generally classified as natural, intensive, and extensive, and the latter type of algal aquaculture pond is for example used with the instant disclosure.

Natural open-algal aquaculture ponds are defined as those naturally occurring algal aquaculture ponds where the conditions are right to grow algae. These algal aquaculture ponds may contain either fresh or saline water, and they are unmanaged in that they lack both controlled fertilizer addition and mechanical agitation. Natural open algal aquaculture ponds that contain algae are common along the shores of the Great Salt Lake in Utah. In this case, the algae would tolerate hypersaline environments.

Both the intensive and extensive modes of aquaculture require the controlled addition of fertilizers to the algal growth medium in order to supply the necessary nutrients, such as phosphorus, nitrogen, iron, and trace metals, that are necessary for algal biomass production through photosynthesis. The primary difference between the two modes of production is mixing of the algal growth medium. Intensive algal aquaculture ponds employ mechanical mixing devices while extensive algal aquaculture ponds rely on natural mixing. Therefore, factors that affect algae growth can be more accurately controlled in intensive aquaculture.

Intensive algal aquaculture ponds are frequently constructed of concrete blocks and are lined with either plastic or clay. Brine depth generally is controlled at about 20-40 centimeters, which has been considered to be the optimum depth for producing algal biomass. A number of configurations of these algal aquaculture ponds have been proposed. However, the open-air raceway algal aquaculture ponds are typically the most important commercially. Raceway algal aquaculture ponds generally employ paddle wheels to provide mixing, although jets may also be used with algae that have cell walls and are thus not as shear sensitive. Chemical and biological parameters are carefully controlled, including salt and fertilizer concentrations, pH of the growth media, and purity of the culture. The drawback with intensive algal aquaculture ponds is the high capital and operating costs for the liners and the mixing equipment. Thus, more efficient modes of mixing and sealing the algal aquaculture ponds are required. Intensive algal aquaculture ponds used to grow Dunaliella salina have been practiced in Calipatria, California by Amway and in Eilat, Israel by Natural Beta Technologies. Cyanotech in Hawaii uses raceway ponds to grow Haematococcus and Spirulina.

Extensive aquaculture has been practiced in the hot and arid regions of Australia for the production of beta-carotene. Outdoor algal aquaculture ponds for extensive aquaculture generally are larger than those for intensive aquaculture and are constructed in coastal lake beds that are located near Walhalla, South Australia and near Hutt Lagoon in Western Australia. Open-air algal aquaculture ponds are typically bounded by earthen dikes. All of these algal aquaculture ponds have been operated in batch mode. In other words, the algal aquaculture ponds are filled with brine and algae, fertilized, and the culture is allowed to grow until it is harvested. The growth media is not transported from algal aquaculture pond to algal aquaculture pond. Also, no mechanical mixing devices are employed.

The algal aquaculture pond(s), e.g., algal aquaculture pond 1, 10, for use in the instant disclosure may be constructed from earth, clay, rock, and combinations thereof, and a majority if not all of the algal aquaculture pond surface area is typically unlined. Characteristics of the soil have been previously described, and it is desirable for the soil to have a low hydraulic permeability. If liners are to be used, they may be deployed at selective minor locations where excessive soil erosion would potentially occur.

The algal aquaculture pond(s) may be any type of algal aquaculture pond used to grow algae, including, but not limited to enclosed bioreactors (such as photoreactors), open algal aquaculture ponds configured either with or without agitation or liners. When present, suitable liner materials include plastic or clay. Plastic algal aquaculture pond liners are typically formed from polyethylene, polypropylene, or polyvinyl chloride. Different types of these basic polymers can be used, for example linear low-density polyethylene liners are occasionally used for algae cultivation at large scale. These liners may also comprise additives, such as carbon black to provide resistance to ultraviolet radiation. These liners may also comprise Nylon or other fibers to provide additional structural integrity. Raven Industries (South Dakota) provides a full line of suitable liners that comprise one or more layers of materials. Suitable clay liners include bentonite clay. However, when algal aquaculture ponds are flooded, components in the water, especially saline, can often form a barrier that seals the algal aquaculture pond. It may also be desirable to include liners in just a portion of the algal aquaculture pond where it is specifically needed. For example, liners may be utilized to protect earthen borders where the hydraulic flow may be elevated.

The state of the art also teaches away from making algal aquaculture ponds larger, as in the present disclosure, with increasing algal aquaculture pond scale. Instead, the art teaches a multiplication of raceway ponds that cover tens of hectares, as was constructed by Qualitas Health in Columbus, New Mexico.

As used herein, the term “about” refers to a value that is ±1% of the stated value. In addition, it is understood that reference to a range of a first value to a second value includes the range of the stated values, e.g., a range of about 1 to about 5 also includes the more precise range of 1 to 5. Further, it is understood that the ranges disclosed herein include any selected subrange within the stated range, e.g., a subrange of about 50 to about 60 is contemplated in a disclosed range of about 1 to about 100.

As used herein, the term “Fetch” 3 refers to the distance, such as an unimpeded distance, that the wind blows across the algal aquaculture pond in the direction of the wind. Fetch can be given or measured in meters. The fetch is needed to mix the algal growth medium. In one embodiment, the Fetch 3 is a dimension of the algal aquaculture pond 1, such as a length, width or diameter of the algal aquaculture pond 1, for example, the longest or the shortest dimension of the algal aquaculture pond 1. In the present disclosure, the Fetch 3 is for mixing the algal growth medium 2 or components thereof within the algal aquaculture pond 1 and/or for circulating the algal growth medium 2 through at least a portion of the algal aquaculture pond 1. Indeed, the Fetch 3 enables creating a degree of turbulence in the algal growth medium 2 of the algal aquaculture pond 1 for mixing the algal growth medium 2 or components thereof within the algal aquaculture pond 1 and/or for circulating the algal growth medium 2 through at least a portion of the algal aquaculture pond 1. For example, in one embodiment when the annual average wind speed 5 is used, then the Fetch 3 in all of the algal aquaculture pond 1 dimensions can be sufficient to cause the algal aquaculture ponds 1 to be mixed.

As discussed herein, Fetch 3 may be used to provide algal aquaculture pond 1 mixing to distribute minerals and nutrients throughout the algal aquaculture pond. This is important in order to maintain a relatively constant salinity throughout the algal aquaculture pond and to distribute nutrients so that the algae grow throughout the algal aquaculture pond instead of in localized regions. Fetch may also be used to mix the algal aquaculture pond in order to enhance carbon dioxide transport into the algal aquaculture pond.

As used herein, the term “wave mixed layer” 7 refers to at least a portion of the algal growth medium 2, in which turbulence is created due to the Fetch 3. The wind creates a surface shear which produces what is sometimes referred to as ‘turbulent kinetic energy’ or TKE. TKE is transported downward in the water column and mixes the algal aquaculture pond 1 to a depth dependent on the wind speed. The depth is approximated by taking one half of the wavelength that is generated by the wind. Put another way, wind-induced surface waves typically have their energy confined to a near-surface layer, whose depth is approximately a half of their wavelength. This near-surface layer is known as a “wave mixed layer.” Mackay, Eleanor B.; Jones, Ian D.; Folkard, Andrew M.; Barker, Philip, 2012, Contribution of sediment focusing to heterogeneity of organic carbon and phosphorus burial in small lakes. Freshwater Biology, 57 (2). 290-304 citing Smith I. R. & Sinclair I. J. (1972), Deepwater waves in lakes. Freshwater Biology, 2, 387-399.

For example, mixing of the algal growth medium 2, mixing the aqueous medium with algal nutrients and/or circulation of the algal growth medium 2 throughout the algal aquaculture pond 1 can be ensured e.g. by controlling the wave mixed layer 7 of the algal aquaculture pond 1 in specific embodiments of the disclosure.

“Algal nutrients” of the algal growth medium 2 disclosed herein may comprise any suitable nutrients that promote the growth of the targeted algae. In an embodiment, the algal nutrients may comprise nitrogen, phosphorus, iron, trace mineral nutrients, and combinations thereof. Suitable nitrogen sources include, but are not limited to ammonia, urea, nitrates, or combinations thereof. Suitable phosphorus sources include, but are not limited to phosphoric acid, diammonium phosphate, phosphates, and other sources of phosphorus. Suitable iron sources are EDTA chelated iron, and other soluble and insoluble forms of iron. There are a number of other micronutrients that are needed by algae, such as sulfur and manganese, copper, zinc, molybdenum and boron that can be provided to the algal growth medium. Many of these micronutrients may at least partially be provided by seawater and other sources of water.

Mixing of only the surface layer of the algal aquaculture pond 1 due to the wave mixed layer 7 is probably sufficient to mix the algal aquaculture ponds 1 to the extent that they need to be mixed, especially if the algal species being cultured are motile. For example, D. salina swim to the surface of the algal aquaculture pond 1 during daylight (or sunlight) and thus, they are mixed if just the surface of the algal aquaculture pond 1 is mixed via the wave mixed layer 7.

The depth and total depth 9 of the algal growth medium 2 in the algal aquaculture pond may be determined by any suitable method. For example, in an embodiment, the total depth 9 of the algal growth medium 2 is measured at two, three or more locations or at a plurality, e.g., 10, 20, 30, 40, 50 or more, of locations in the algal aquaculture pond 1 and a mean total depth is determined. Thus, in an embodiment, the depth of the algal growth medium in the algal aquaculture pond is a mean depth of the algal growth medium in the algal aquaculture pond. In certain embodiments, the mean depth of the algal growth medium 2 is based on the depth throughout the entire algal aquaculture pond area. The mean depth of the algal growth medium can be given or measured in meters. In one embodiment, e.g. in cases where the bottom of the algal aquaculture pond 1 is flat or about flat, the depth of the algal growth medium 2 is measured at only one location in the algal aquaculture pond 1. It is most desirable for the algal aquaculture pond 1 not to have any shallow areas that can protrude from the surface of the water. Ideally, the algal aquaculture pond 1 would be relatively flat, and not have high areas that would segment the algal aquaculture pond 1, but that is not essential. Ideally, the algal aquaculture ponds 1 can be laser leveled and/or can have a slight slope towards the drain.

In certain embodiments, the depth of the algal growth medium 2 in the algal aquaculture pond 1 is less than the wave mixed layer 7. In this way, adequate mixing of components of the algal growth medium 2 can be achieved in specific embodiments. Mixing of the algal growth medium 2, mixing the aqueous medium with the algal nutrients and/or circulation of the algal growth medium 2 throughout the algal aquaculture pond 1 may be ensured e.g. by controlling the depth of the algal growth medium 2 in specific embodiments of the disclosure. In certain embodiments, the depth of the algal growth medium 2 in the algal aquaculture pond 1 is from about 0.15 meters to about 2 meters.

In certain embodiments, the ratio of the wave mixed layer 7 of the algal growth medium 2 to the depth of the algal growth medium 2 in the algal aquaculture pond 1 is greater than about 0.3, greater than about 0.4, greater than about 0.5, greater than about 0.6, greater than about 0.7, greater than about 0.8, greater than about 0.9 or even greater than about 1.0. In particular embodiments, the ratio is about or greater than 1.0. In certain embodiments, the ratio is from about 0.5 to about 2.0, such as greater than about 1.0 to about 2.0. By having a ratio of the wave mixed layer of the algal growth medium 2 to the depth of the algal growth medium 2 in the algal aquaculture pond 1 greater than above about 1.0, components of the algal growth medium 2 are ensured to be adequately mixed and circulated throughout the algal aquaculture pond 1. However, by having this ratio above 1.0, there is potential for sediment to be suspended from the bottom of the pond that leads to turbidity of the algal aquaculture medium. Turbidity is typically undesired, so in some exemplary embodiments the type of soil from FIG. 3 can be important in determining the proper fetch of the ponds.

In another exemplary embodiment, sufficient mixing of the algal aquaculture pond 1 may occur when the ratio of the wave mixed layer 7 divided by the mean algal aquaculture pond depth is greater than about 0.5 because conservation of mass causes undercurrents in the algal aquaculture pond 1 to transport media in the opposite direction of the wind-thus mixing the algal aquaculture pond 1. The mixing in this configuration (22, 24) is shown in FIG. 2. This case assumes no slip at either the gas-liquid interface or the solid-liquid interface at the bottom of the algal aquaculture pond 1. In this embodiment, the wave mixed layer 7 (FIG. 1) only mixes the top half of the algal aquaculture pond depth, and the circulatory flow mixes the bottom half of the algal aquaculture pond 1.

In addition, in certain embodiments, at least a portion of algal nutrients are intentionally added to an aqueous stream to form the algal growth medium 2, thus meaning the algal growth medium 2 is also not naturally occurring in certain embodiments.

Thus, in one exemplary embodiment, the algal aquaculture pond 1 or system comprises any suitable structure(s) for adding algal nutrients to an aqueous medium or a stream to form the algal growth medium 2. In an exemplary aspect, the algal aquaculture pond 1 or system comprises a canal or a conduit for feeding the algal growth medium 2 or an aqueous medium or a stream to the algal aquaculture pond 1.

The present disclosure overcomes the deficiencies of the prior art by the following disclosure of processes, systems, mixed algal aquaculture medium, algal aquaculture ponds, and uses thereof, for culturing algae, which advantageously utilizes “Fetch” as described herein, for the mixing of and/or circulation of components, e.g., algal nutrients in an algal growth medium, instead of mechanical means. By utilizing Fetch compared to traditional mechanical equipment to mix the components of the algal growth medium and/or to circulate the algal growth medium throughout the algal aquaculture pond, large-scale growth of algae is enabled whilst significantly reducing capital and operating costs. This provides a significant commercial advantage for culturing algae.

The reduced capital is also partly accomplished by changing the scale-up approach to making each algal aquaculture pond larger than those typically used, instead of using a greater number of the same size algal aquaculture ponds. The present approach, utilizing controlled and specific properties of the algal aquaculture pond and in the algal aquaculture pond enables production of algae in large quantities at a large scale and with low energy consumption. Large algal aquaculture ponds are significantly less costly to construct than a multiplicity of raceway ponds due to the elimination of: 1) power lines connected to every paddlewheel, 2) transfer pumps and lines from each pond to the harvester, 3) capital associated with the paddlewheels and motors, and 4) nutrient delivery systems to each raceway pond. Instead, large algal aquaculture ponds using fetch to mix the ponds have the following advantages: 1) they operate in continuous flow by gravity throughout the system that does not require pumps; 2) power lines do not need to be run throughout the aquaculture system; 3) by using continuous flow, nutrients can be supplied to the entire aquaculture system from a single source; and 4) many capital cost items such as pumps, paddlewheels, motors, and liners can be eliminated.

In an embodiment, the “flow” of algal growth medium 2 disclosed herein and/or components for the algal growth medium 2 are flowed continuously into the algal aquaculture pond 1 as algal growth medium 2 is removed from the algal aquaculture pond 1. In this way, the algal aquaculture pond 1 can be operated continuously. In one embodiment the flow of the algal growth medium 2 and/or components for the algal growth medium 2 is (are) continuously added to the algal aquaculture pond 1 to replace evaporative losses and loss from intentional removal of media to harvest the algae. In one embodiment, the algal aquaculture pond 1 is a continuous flow type growth reactor.

Thus, in an exemplary aspect there is provided, the process comprising: the open algal aquaculture pond 1 being configured to continuously remove the algal growth medium 2 from the open algal aquaculture pond 1.

In another exemplary embodiment, to facilitate the continuous flow of algal growth medium 2 into and out of the algal aquaculture pond 1 and/or the cleaning of the algal aquaculture pond 1, the algal aquaculture pond 1 may have a degree of slope along a bottom of the algal aquaculture pond 1 from an inlet end to an outlet end of the algal aquaculture pond 1 (bottom slope). In an embodiment, the bottom slope is at least about 5 cm, such as about 5-about 15 cm or even more, per 1000 meters of length of the algal aquaculture pond 1.

In one embodiment, the wave mixed layer 7 may be computed with the mean wind speed 5 and Fetch 3. The mean wind speed (e.g. 5, FIG. 1) used in this calculation can be defined e.g. as the annual mean wind speed at the algal aquaculture pond 1 site or at the nearest airport where the mean wind speed is measured. Typically, the units of the mean wind speed is in meters per second. In the case when algae only grow for a portion of the year at a given location, then the mean wind speed used in the calculation can be e.g. that for the months when algae are growing. The wind speed can be taken or measured at a certain height over the water surface, typically e.g. at about 2 meters height or at a height lower or higher than about 2 meters over the water surface.

Thus, in an exemplary aspect of the present process there is provided, the process wherein the WML 7 is selected as a function of a constant, a specified wind speed 5, and a specified fetch 3.

In another exemplary aspect there is provided, the process wherein the fetch 3 is specified as an average distance wind will travel across the open algal aquaculture pond 3.

In another exemplary aspect there is provided, the process wherein the fetch 3 is the length of the algal aquaculture pond 1 in a direction of wind travel.

In certain embodiments, the Fetch 3 may range from about 75 to about 3000 meters or from about 80 to about 2000 meters, or more preferably from about 100 to about 1500 meters optionally across a longest dimension of the algal aquaculture pond. Further, the Fetch may be in a direction transverse to the longest dimension of the algal aquaculture pond. The Fetch of an algal aquaculture pond may be adjusted by installing floating breakwaters across the algal aquaculture pond or floating nets on the surface of the algal aquaculture pond.

In certain embodiments, the Fetch 3 may be selected for a given pond based upon the windrose which shows wind direction, wind speed, and probability. These parameters are used to compute the Fetch based on the windrise data.

Thus, in another exemplary aspect there is provided, the process wherein the fetch 3 is a distance selected to be at least one or more of about 75 to about 3000 meters, from about 80 to about 2000 meters, and/or from about 100 to about 1500 meters, optionally across the longest dimension of the algal aquaculture pond 1.

In another exemplary aspect there is provided, the process wherein the ratio of the WML 7 of the algal growth medium 2 to the total depth 9 of the algal growth medium 2 in the algal aquaculture pond 1 is selected to be at least one or more of greater than about 0.3, about 0.4, about 0.5, about 0.6, about 0.7, about 0.8, about 0.9, and/or about 1.0.

In another exemplary aspect there is provided, the process wherein the algal growth medium 2 comprises algal nutrients such as added algal nutrients.

In another exemplary aspect there is provided, the process wherein the total depth 9 of the open algal aquaculture pond 1 is selected to be at least one or more of variant and/or consistent.

In another exemplary aspect there is provided, the process wherein the total depth 9 of the algal aquaculture pond 1 aids the WML 7 to promote mixing, and/or the total depth 9 at any relative position is from about 0.15 m to about 4 m. For example, from about 0.15 m to about 2 m. In one embodiment, in a season when the mean wind speed is high, the depth of the pond may be increased to reduce the amount of turbidity due to the suspension of particles, if the WML is greater than about unity. In yet another embodiment, in a season when the mean wind speed is low, the pond depth may be lowered in order to achieve a greater ratio of WML to pond depth.

In an exemplary embodiment, the algal aquaculture pond 1 is capable of producing and maintaining an algal concentration of about 20,000 to about 2,000,000 algal cells per milliliter averaged over a top (about) 15-(about) 30 cm of a depth of the algal growth medium or over a top (about) 10% of a depth of the algal growth medium. In further embodiments, dissolved inorganic carbon or a mass transfer of carbon dioxide satisfies an algal concentration of about 20,000 to about 2,000,000 cells per milliliter averaged over the top (about) 15-(about) 30 cm of a depth of the algal growth medium or over a top (about) 10% of a depth of the algal growth medium. Dissolved inorganic carbon comprises carbon dioxide and bicarbonate/carbonate. In yet another exemplary embodiment, the algal aquaculture pond 1 is capable of producing and maintaining an algal concentration of about 20,000 to about 2,000,000 algal cells per milliliter averaged throughout the water column.

In an exemplary aspect there is provided, the process wherein the open algal aquaculture pond 1 contains an average algal cell concentration of about 20,000 to about 2,000,000 algal cells per mL.

In another exemplary aspect there is provided, the process wherein the open algal aquaculture pond 1 is an open algal aquaculture pond 1 of a selected volumetric capacity for optimal mixing.

In another exemplary aspect there is provided, the process wherein the open algal aquaculture pond 1 includes at least one dimension larger than 50 m, and/or a mean depth of less than 4 m.

In an exemplary embodiment, the size of the algal aquaculture pond 1 is from about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, about 5-about 10 hectares, or about 100-about 1000 hectares.

In an exemplary aspect there is provided, the process wherein the open algal aquaculture pond 1 has an algal aquaculture pond size selected to be at least one or more of greater than about 10, about 20, about 50, about 100 hectares, and/or up to about 1000 hectares; optionally wherein the size of the algal aquaculture pond is from about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, about 5-about 10 hectares, or about 100-about 1000 hectares.

Based on the mixing data collected on an algal aquaculture pond 1 with a size greater than about 150 hectares as presented in the Examples, the ratio of the wave mixed layer divided by the mean algal aquaculture pond depth is greater than about 0.9 in order to mix the contents of the algal aquaculture ponds 1. In this embodiment, the wave mixed layer 7 was occasionally calculated to be greater than the total algal aquaculture pond depth as well. Thus, in this embodiment, the convective mixing in the wave mixed layer 7 penetrates the entire total algal aquaculture pond depth 9.

In certain embodiments, due to the Fetch 3 enabling the necessary energy for mixing and circulating of the algal growth medium 2 in the algal aquaculture pond 1, the algal aquaculture pond 1 does not comprise one or more mechanical mixing device(s). This saves significant operating and capital costs associated with providing necessary equipment to otherwise achieve these aims.

In another exemplary aspect there is provided, the process comprising: utilizing mechanical means for mixing, or not including a step of utilizing mechanical means for mixing. For example, although not necessary the mixing may be aided by utilizing additional mechanical means for mixing.

In another exemplary aspect there is provided, the process wherein the open algal aquaculture pond 1 is built on a foundation with permeability for holding algal growth medium 2 containing algae whilst facilitating the wind (or the WML) 7.

In another exemplary aspect there is provided, the process wherein the foundation includes clay, rock, or concrete, or a combination thereof.

In another exemplary aspect there is provided, the process wherein the open algal aquaculture pond 1 has a non-uniform cross-section.

In another exemplary aspect there is provided, the process wherein the open algal aquaculture pond 1 has a configuration that is selected to be at least one or more of circular, oval, square, triangular, trapezoidal, and/or rectangular, or any combination thereof. In another exemplary aspect, the partially manmade structure or configuration of the open algal aquaculture pond 1 is designed to fit the local topology in order to minimize the amount earthen work required to construct the algal aquaculture ponds. In yet another exemplary aspect, the partially manmade structure or configuration of the open algal aquaculture pond 1 is that of a raceway. For example, a trapezoidal raceway, as would be understood by one of ordinary skill in the art, or a horizontal trapezoidal raceway.

The wave mixed layer 7 of the algal growth medium 2 in the algal aquaculture pond 1 may be any suitable amount. In an embodiment, the wave mixed layer 7 may be greater than 1.5 cm such as greater than 2.5 cm.

The Wave Mixed Layer (WML) is a function of Fetch and Wind Speed as expressed in the equations below.

$$Ln ( WML ) = - 0.18691 + 0 \text{.48936} * Ln ( Fetch ) + 1.0365 * Ln ( Wind Speed ) \qquad Equation ( 1 )$$

$$WML = Exp [ - 0.18691 + 0 \text{.48936} * Ln ( Fetch ) + 1.0365 * Ln ( Wind Speed ) \qquad Equation ( 2 )$$

$$WML = e [ - 0.18691 + 0 \text{.48936} * Ln ( Fetch ) + 1.0365 * Ln ( Wind Speed ) ] \qquad Equation ( 3 )$$

$$Where e = 2.718281284 , and WML is the Wave mixed layer$$

$$Or approximately : WML = 0.83 ( Wind Speed ) ( Sq . Root of Fetch ) \qquad Equation ( 4 )$$

In the above equations, Fetch (3, FIG. 1) is measured in meters, Wind Speed (5, FIG. 1) is in m/sec and WML (7, FIG. 1) is in cm.

In certain embodiments, the wave mixed layer (WML) 7 of the algal growth medium 2 is calculated according to the equation (5) below:

$$WML = {e}^{[ - 0.18691 + 0.48936 * Ln ( Fetch ) + 1.0365 * Ln ( Wind Speed ) ]} \qquad Equation ( 5 )$$

$$where e = 2.7182818284 and WML is the wave mixed layer in centimeters .$$

The wind speed (5, FIG. 1) is measured in m/sec and the Fetch (3, FIG. 1) is measured in meters.

Algal growth is a function of the area of the algal aquaculture pond on which sunlight falls. Algae in the upper most layer of the algal aquaculture pond intercept sunlight and use it to convert carbon dioxide in their growth process. The uppermost layer of algae may block sunlight from reaching algae at lower depths. It is most desirable that the uppermost layer has sufficient carbon dioxide so that algal growth is not limited. Equations 1-3 depict how WML varies with Fetch and Wind Speed. It is desirable to have a WML of 2.5 cm or greater. This may be achieved through any suitable combination of Fetch and Wind Speed according to Equations (1)-(3).

In one embodiment, if the minimum requirement for WML is met, algal aquaculture ponds need not be aligned with the predominant wind direction but it is preferable to do so. The growth zone of an algal aquaculture pond or algal aquaculture ponds is that section in which the minimum WML requirements are met.

Thus, in a particular embodiment, the wave mixed layer 7 in the algal aquaculture pond 1 is greater than about 1.5 cm or greater than about 2.5 cm according to equation (5).

In another exemplary aspect there is provided, the process wherein the WML 7 is obtained using WML=0.830 (wind speed) (√fetch), wherein 0.830 is the constant.

In another exemplary aspect there is provided, the process wherein one or more algal nutrients are applied to the algal growth medium 2 for optimal algal growth.

In another exemplary aspect there is provided, the process wherein the one or more algal nutrients are applied in the form selected to be at least one or more of a liquid solution, as a slurry, as a solid, and/or as machine processed pellets, or any combination thereof.

The processes disclosed herein comprise a surface temperature of the algal aquaculture pond 1 that is acceptable for algal growth.

In another exemplary aspect there is provided, the process wherein the WML 7 is measured by e^{[-0.18691+0.48936*In(fetch)+1.0365*In(wind speed)]}.

In another exemplary aspect there is provided, the process according to any preceding claim, wherein the open algal aquaculture pond 1 contains a constant carotene/chlorophyll ratio. This is especially important to control and maintain high product quality and consistency.

In addition, since the algal aquaculture pond 1 is utilized for growing algae, in certain embodiments, the algal aquaculture pond 1 comprises algae therein.

For example, the algae for growth in the algal aquaculture pond may comprise any suitable algae capable of growing in an algal growth medium. Without limitation, algae from the divisions Chlorophycophyta, Phaeophycophyta, Chrysophycophyta, Cyanophycophyta, Cryptophycophyta, Pyrrhophycophyta and Rhodophycophyta, which are adaptable to saline water as an algal growth medium, are all suitable for use in the present disclosure.

A more preferred group of algae are those with flagella, cilia and/or eyespots. Flagella are a tail-like projection that protrudes from the cell body of certain algae and functions in locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g., Dunaliella) can perceive light by means of a sensitive eyespot and move to regions of higher light concentration to enhance photosynthesis.

The algae may be any suitable type grown in an algal growth medium 2. In an embodiment, the algae are selected from microalgae, phototaxic microalgae, or one or more of the following algae: Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., and genetically-engineered varieties or combinations (mixtures, or mixed cultures) thereof; and one or more of the following microalgal species including Amphora sp., Ankistrodesmus, Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schiochytrium limacinum, Stichococcus sp., Tetraselmis suecica, and Thalassiosira pseudonana, and genetically-engineered varieties or combinations (mixtures, or mixed cultures) thereof. In an embodiment, the algal biomass comprises algae exhibiting phototaxis, such as the algae Dunaliella.

In one exemplary embodiment, the algae or microalgae discussed herein have not been genetically modified or do not originate from genetically engineered algae or microalgae. In a specific embodiment, the algae or microalgae is selected from the group comprising or consisting of Dunaliella sp., Dunaliella bardawil, Dunaliella salina, Dunaliella tertiolecta, Dunaliella parva and Dunaliella viridis, and any combination thereof. In a specific embodiment, the algae or microalgae is Dunaliella salina.

In another exemplary aspect there is provided, the process wherein the open algal aquaculture pond 1 comprises algae selected to be at least one or more of: Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia losterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and/or genetically-engineered varieties, or any combination thereof.

In certain embodiments, the algal aquaculture pond 1 comprises or may be utilized to grow hypersaline algae, i.e., algae grown in a medium 2 having a salinity of at least about 7 wt % such as at least about 10 wt %. Thus, in certain embodiments, the salinity of the algal growth medium 2 is at least about 7 wt %, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt % (e.g., up to saturation).

In particular embodiments, the salinity of the algal growth medium 2 is from about 7 wt % to saturation, from about 8 wt % to saturation, from about 9 wt % to saturation, from about 10 wt % to saturation, from about 20 wt % to saturation, about 10 wt % to about 25 wt %, about 10 wt % to about 20 wt %, about 10 wt % to about 15 wt %, about 12 wt % to about 25 wt %, about 15 wt % to about 25 wt %, or about 20 wt % to about 25 wt %. In certain embodiments, the algal growth medium is saturated with salt. The salinity of the algal growth medium may comprise any suitable salts for providing the desired salinity. In an embodiment, the salinity comprises sea salts, underground salts, salts of aquifer water, salts of a terminal lake, sodium chloride, and/or any combination of ions present in sea salt.

In an exemplary aspect there is provided therefore, the process wherein the algal growth medium 2 contains a salinity from about 5 wt % to about 27 wt %.

As used herein, “wt %” refers to a dry mass of a component in a solution in grams divided by 100 grams of the solution. In addition, unless otherwise stated herein or clear from the context, any percentages referred to herein are understood to refer to wt %.

In another exemplary aspect there is provided, a continuous flow algal culturing system for mixing an algal growth medium 2, the system comprising:
at least one or more of an open algal aquaculture pond 1, the open algal aquaculture pond 1 possessing a fetch 3 selected as a function of a specified wind speed 5 and a wave mixed layer (WML) 7 defined by a specified depth from the surface of the algal aquaculture pond 1; at least a part of the open algal aquaculture pond 1 having at least partially a manmade configuration and being in communication with a harvester (e.g. 314, FIG. 5) for separating algal biomass from the algal growth medium; and wherein a ratio of the WML 7 of the algal growth medium 2 to a total depth 9 of the algal growth medium 2 in the algal aquaculture pond 1 is greater than about 0.2.

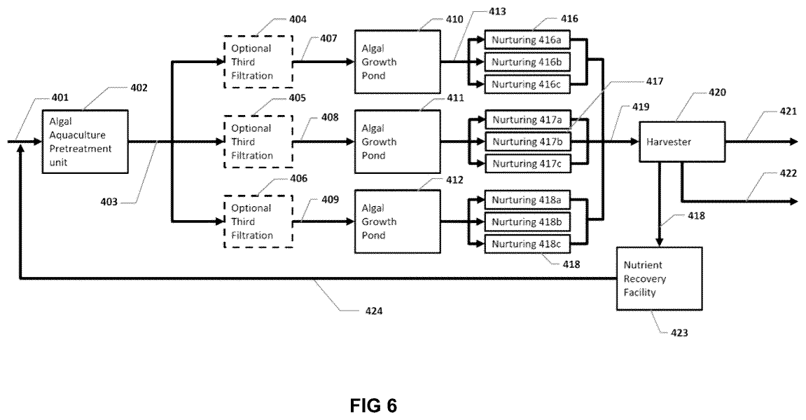

In another exemplary aspect there is provided, the system comprising: at least one device to a) monitor, supply and/or maintain target nutrient concentrations for optimal algal growth, and/or b) monitor, supply and/or maintain average concentrations of the algae and the nutrients at greater than or equal to their concentrations in an effluent flow. For example, the at least one device being in communication with the at least one open algal aquaculture pond (e.g. the at least one algal growth pond 410, FIG. 6) as disclosed herein.

In another exemplary aspect there is provided, the system wherein the ratio is selected to be one or more of greater than about 0.3, about 0.4, about 0.5, about 0.6, about 0.7, about 0.8, about 0.9, and/or about 1.0. In a further exemplary aspect, is the system described herein comprising the ratio selected to be one or more of greater than about 1.1, about 1.2, about 1.3, about 1.4 about 1.5, about 1.6, about 1.7, about 1.8, about 1.9, and/or about 2.0. In a particular aspect, the ratio is between about 0.3 and 2.0.

In another exemplary aspect there is provided, the system consisting of a plurality of interconnected algal aquaculture ponds 1 containing selected fetches 3.

In another exemplary aspect there is provided, the system wherein the open algal aquaculture pond 1 is connected to at least one algal pre-treatment unit (e.g. 402, FIG. 6) and/or at least one extraction unit.

In another exemplary aspect there is provided, the system wherein the harvester (e.g. 314, FIG. 5) includes at least one or more of: an adsorptive bubble separation unit, a centrifugation unit, a flocculation unit, a sedimentation unit, and/or a filtration unit, or any combination thereof.

In another exemplary aspect there is provided, a mixed algal aquaculture medium (e.g. 2_{mixed}, FIG. 1) obtainable by a process disclosed herein.

There is provided a use of any embodiment of an algal aquaculture pond 1 or system for growing or cultivating algae as described or otherwise encompassed by the present description.

In a further exemplary aspect there is provided, use of a mixed algal aquaculture medium (e.g. 2_{mixed}, FIG. 1), or a system as described herein, for cultivating or culturing algae.

In an alternative exemplary aspect, there is also disclosed an algal aquaculture pond 1 for growing algae comprising an algal growth medium 2 comprising algal nutrients (e.g. added algal nutrients) within the algal aquaculture pond 1; and a Fetch 3 capable of mixing the algal growth medium 2 and/or circulating the algal growth medium 2 throughout the algal aquaculture pond 1, wherein the ratio of a wave mixed layer 7 of the algal growth medium 2 to a total depth 9 of the algal growth medium 2 in the algal aquaculture pond is greater than about 0.2.

In a further exemplary aspect, there is also disclosed an algal aquaculture pond 1 for cultivating algae comprising an algal growth medium 2 containing algal nutrients (e.g. added algal nutrients) within the algal aquaculture pond 1; and a Fetch 3 capable of mixing the algal growth medium 2 and/or circulate the algal growth medium 2 throughout the algal aquaculture pond 1, wherein the ratio of a wave mixed layer 7 of the algal growth medium 2 to a total depth 9 of the algal growth medium 2 in the algal aquaculture pond 1 is greater than about 0.2.

In an alternative exemplary aspect, there is provided a system for growing or cultivating algae, wherein the system comprises one algal aquaculture pond 1, two algal aquaculture ponds or a plurality of algal aquaculture ponds as described or otherwise encompassed by the present description.

There is also provided a process for growing or cultivating algae, wherein the process comprises growing or cultivating algae in any embodiment of an algal aquaculture pond 1 or system as described or encompassed by the current description, and optionally harvesting the grown or cultivated algae.

There is also provided herein a process for growing algae comprising:
determining a predominant wind direction from historical wind direction data for a predetermined area; based on the predominant wind direction, providing an algal aquaculture pond with: (i) a Fetch 3 capable of mixing an algal growth medium 2 in the algal aquaculture pond and/or circulating the algal growth medium throughout the algal aquaculture pond, and (ii) a ratio of a wave mixed layer 7 of the algal growth medium 2 to a total depth 9 of the algal growth medium 2 in the algal aquaculture pond 1 of greater than about 0.2; and growing algae in the algal aquaculture pond 1.

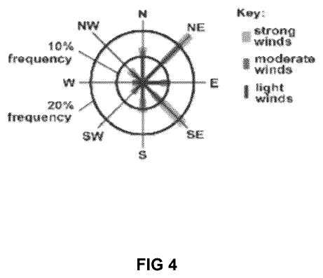

The algal aquaculture pond 1 comprises constructing the algal aquaculture pond 1 such that a longest dimension of the algal aquaculture pond 1 is arranged in the determined predominant wind direction for the predetermined area. For example, a longest dimension of one algal aquaculture pond 1, two algal aquaculture ponds or each of the algal aquaculture ponds is arranged in the predominant wind direction as described herein are disclosed. Such as in the wind directions as shown in FIG. 4.

In one embodiment, the process further comprises determining a predominant wind direction from wind direction data for a predetermined area; and arranging a longest dimension of the algal aquaculture pond 1 or one, two, or each of the algal aquaculture ponds in the determined predominant wind direction within the predetermined area. In one embodiment of the process, the arranging of the longest dimension of the algal aquaculture pond 1 or one, two, or each of the algal aquaculture ponds in the predominant wind direction enables or provides mixing the algal growth medium 2 and/or circulating the algal growth medium 2 throughout the algal aquaculture pond 1.

In one embodiment, the process further comprises providing the algal aquaculture pond 1 with an algal growth medium 2 for the growth of the algae in the algal aquaculture pond 1, the algal growth medium 2 comprising an aqueous medium and algal nutrients.

In a further exemplary embodiment, to optimize the turbulence created by the Fetch 3, and thus mixing of the components within the algal growth medium 2 and circulation of the algal growth medium 2 throughout the algal aquaculture pond 1, the longest dimension of the algal aquaculture pond 1 may be arranged in a predominant wind direction for an area encompassing the algal aquaculture pond 1.

FIG. 2, for example, illustrates an algal aquaculture pond 10 or a system of the present disclosure comprising an algal aquaculture pond 10 having an algal growth medium 12 therein and a longest dimension 14 within an area 16 that encompasses the algal aquaculture pond 10. There is further shown (by reference numeral 22) a predominant wind direction for area 16 encompassing the algal aquaculture pond 10.

The area 16 is at least as large as the algal aquaculture pond 10 itself. In the case of a plurality of algal aquaculture ponds as the type of algal aquaculture pond 10, the area 16 is at least as large as the area that encompasses all of the algal aquaculture ponds 10. In addition, in certain embodiments, the area 16 for which a predominant wind direction is direction includes the algal aquaculture pond 10 or a plurality of such algal aquaculture ponds 10, and optionally no more than 10×, 5×, 3×, or 2× the area of such algal aquaculture pond 10 or algal aquaculture ponds 10.

Further, by “longest dimension of the algal aquaculture pond,” it is thus meant a line, e.g., line 14 having a maximum distance in the algal aquaculture pond 10 in a line of sight between a single point on one end 18 of the algal aquaculture pond to a single point on an opposite end 20 of the algal aquaculture pond 10 as shown in FIG. 2. It is contemplated that the predominant wind direction 22 may not be represented by a single line, but rather a plurality of lines, i.e., a section or segment of a circle.

In certain embodiments, when the algal aquaculture pond 10 is constructed, the algal aquaculture pond 10 is arranged such that the longest dimension 16 is arranged within about 30 degrees, about 25 degrees, about 20 degrees, about 15 degrees, about 10 degrees, or within about 5 degrees of the predominant wind direction 22 for the algal aquaculture pond 10. By “arranged within,” it is meant that at least one straight line, line 24, traveling through the determined predominant wind direction (i) is directly in line with the line 14 representing the longest dimension of the algal aquaculture pond 10 (FIG. 2); or (ii) is within a certain angle (shown as e) (e.g., within about 30 degrees, about 25 degrees, about 15 degrees, about 10 degrees, or about 5 degrees) relative to the line 14 representing the determined predominant wind direction as shown in FIG. 2.

In certain embodiments, the predominant wind direction is determined by historical wind direction data optionally for a predetermined duration, such as at least about 6 months, at least about 1 year, at least about 2 years, at least about 5 years, or at least about 10 years. The historical wind direction data may be received, collected, and provided by any suitable method known in the art. See FIG. 4 for exemplary wind directions and wind strengths such as strong, moderate and light wind speeds.

In an embodiment, for example, the wind direction data comprises wind rose data optionally for a predetermined duration. Wind rose data is a well-known graphical representation of wind conditions, direction and speed, over a period of time for a specific area. Typically, to provide such wind rose data, average wind direction and wind speed values are measured for an area, at short intervals, over a predetermined duration. Thereafter, the collected data are sorted by wind direction such that the percentage of time that the wind was blowing from each direction is determined. In the standard wind rose, a line segment is drawn in each of perhaps eight compass directions from a common origin (see FIG. 4). The length of a particular segment is proportional to the frequency with which winds blow from that direction. Parts of a given segment are given various thicknesses, indicating frequencies of occurrence of various classes of wind speed from the given direction.

In some instances, the wind direction data is sorted into twelve equal arc segments, such as 30° arc segments, in preparation for plotting a circular graph in which the radius of each of the twelve segments represents the percentage of time that the wind blew from each of the twelve 30° direction segments. Wind speed data can be superimposed on each direction segment to indicate, for example, the average wind speed when the wind was blowing from that segment's direction and the maximum wind speed during the logging period.

In an embodiment, the predetermined duration for the wind rose data is at least about 6 months, at least about 1 year, at least about 2 years, at least about 5 years, or at least about 10 years. In this way, the determined predominant wind direction is based upon long-term conditions for the area encompassing the algal aquaculture pond(s) 10.

In the case when additional wind data are available, such as that reported in the style of a wind rose, then that data can be used efficiently to design the algal aquaculture pond or system of the present disclosure. Specifically, a wind rose typically shows both the wind speed in several directions and the probability that the wind will blow in that direction at that speed. This information can be used to compute the required Fetch to mix the algal aquaculture pond when the wind blows in that direction. In this case, there will be a minimum design velocity (speed in that direction) above which the algal aquaculture pond will be mixed. Thus, the minimum Fetch in that direction can be computed. Therefore, the minimum Fetch 3 in each direction can be computed so that the minimum dimension of the algal aquaculture pond 1 can be determined in order to achieve mixing with a probability that is greater than the probability that the wind is blowing above a minimum velocity. Therefore, the algal aquaculture pond 1 dimensions needed to mix the contents of the algal aquaculture pond 1 for the desired percentage of time are determined based on Fetch 3. In certain embodiments, at least one algal aquaculture pond (e.g. 1, 10; FIG. 1 and FIG. 2) is constructed based on historical wind direction data for an area encompassing the algal aquaculture pond 1 to provide an amount of Fetch 3 capable of growing algae in the algal aquaculture pond 1.

In an exemplary embodiment, the systems, processes, and uses encompassed by the current description may include a single algal aquaculture pond 10 (FIG. 2), two or more such algal aquaculture ponds 10 or a plurality of such algal aquaculture ponds 10. The use of a plurality of algal aquaculture ponds 10 allows for further reducing capital and operational costs by utilizing Fetch 3 (FIG. 1) to induce turbulent mixing of the components of the algal growth medium 12 and circulation of the algal growth medium 12 throughout each of the algal aquaculture ponds 10.

In an embodiment, the predominant wind direction represents a predominant wind direction during daylight hours. In this way, the predominant wind direction is determined during periods when wind speeds are expected to be at a maximum.

In an embodiment, an angle of the walls of the algal aquaculture pond 1 are equal to or less than an angle of repose for the material of the walls of the algal aquaculture pond 1. In this way, the walls of the algal aquaculture pond 1 may keep their shape and without the aid of expensive liners or the like. The proper slope of the wall will minimize erosion caused by wind and wave action. In other embodiments, the algal aquaculture pond 1 is surrounded by one or more dikes. In particular embodiments, the algal aquaculture pond 1 further comprises one or more dikes at an outlet end of the algal aquaculture pond 1 to minimize losses of the algal growth medium 2, and algae, when present, from the algal aquaculture pond 1. Optionally, the one or more dikes may comprise a freeboard to minimize losses of the algal growth medium 2 and algae from the algal aquaculture pond 1.

In certain embodiments, at least a part of the algal aquaculture pond 1 is surrounded by one or more berms with a load bearing capacity for vehicular traffic. In this way, travel may be made about the algal aquaculture pond 1 without disturbing the structural integrity of the algal aquaculture pond 1.

The algal aquaculture pond 1 may comprise any suitable structure(s) for allowing discharge of the algal growth medium 2 from the algal aquaculture pond 1. In certain embodiments, the algal growth medium 2 may be discharged to an open body of water, such as an ocean or river. In other embodiments, following growth of the algae in the algal growth medium 2 in the algal aquaculture pond 1, the algal growth medium 2 may be discharged to further growth algal aquaculture ponds 1 and/or one or more harvesters in fluid communication with the algal aquaculture pond 1. In certain embodiments, the algal aquaculture pond 1 comprises one or more weirs for flow of the algal growth medium 2 thereover or thereunder and/or a drain to permit partial or complete withdrawal of the algal growth medium from the algal aquaculture pond. In one embodiment the algal aquaculture pond 1 comprises an exit-weir for overflow of the algal growth medium.

In certain embodiments, a quiescent zone may be provided in the algal aquaculture pond to facilitate harvesting of the algae from the algal aquaculture pond. In certain embodiments, a quiescent zone may be provided in proximity to the inlet side of a weir in order to allow phototaxis of algae to move them closer to the surface (gas-liquid interface) to facilitate enhanced discharge of the algae over the top of the weir. The quiescent zone may be facilitated by one or more techniques, such as erecting barriers to wind in the algal aquaculture pond near the weir to reduce wind-driven mixing. The quiescent zone may also be facilitated by increasing the depth of the algal aquaculture pond in order to reduce mixing due to Fetch. The distance from the weir that the algal aquaculture pond depth needs to be increased will depend on the anticipated flowrate of the media, and the velocity at which algae may swim towards a surface light. For example, it has been reported by Nielson (U.S. Pat. No. 4,958,460) that D. salina can move at a rate of 0.15 feet per hour. Thus, in the present disclosure, one way to enhance the phototaxis is to add a light to a weir box so that the algae swim to the light, even during the night. This is a preferred method of generating a quiescent zone because the addition of structures inside the algal aquaculture pond to block wind are difficult to maintain. Nevertheless, in certain embodiments, the algal aquaculture pond may comprise structures designed to block the wind.

Thus, in certain embodiments, the algal aquaculture pond 1 may further comprise one or more quiescent zones in the algal aquaculture pond 1 to provide an area of reduced turbulence and mixing of the algal growth medium 2 relative to other portions of the algal aquaculture pond 1. This allows the settling and harvesting of the algae from the one or more quiescent zones. In an embodiment, the algal aquaculture pond 1 comprises a quiescent zone at an end of the algal aquaculture pond 1, such as at an exit-weir end of the algal aquaculture pond 1, to facilitate harvesting and the removal of algae from the algal aquaculture pond 1.

In an embodiment, the quiescent zone is formed by providing a section of the algal aquaculture pond 1 with a greater depth than an adjacent portion of the algal aquaculture pond 1. In this way, the wave mixed layer 7 extends to a lesser degree into the depth of the algal aquaculture pond 1 and less mixing/turbulence is seen in the quiescent zone. In an embodiment, the quiescent zone has a depth of at least about 0.5 meters, at least about 1 meter, at least about 2 meters, at least about 3 meters, or at least about 4 meters.

In particular embodiments, the quiescent zone comprises a depth gradient extending from a mean depth of the algal growth medium in the algal aquaculture pond to a deeper depth, e.g., to a depth of about 0.5-about 5 meters, about 0.5-about 4 meters, about 0.5-about 3 meters, or about 0.5-about 2 meters, such as about 1-about 4 meters.

In another aspect, there is provided a system for cultivating algae, wherein the system comprises one, two or a plurality of algal aquaculture ponds 1 as described or otherwise encompassed by the present description. In an embodiment, a longest dimension of one algal aquaculture pond, two algal aquaculture ponds, or each of the algal aquaculture ponds, such as each of the plurality of the algal aquaculture ponds, is arranged in the predominant wind direction as described herein.

In certain embodiments, the algal aquaculture pond 1 or system as described herein may further comprise one or more harvesters (314, FIG. 5) for harvesting algae in fluid communication with the one or more algal aquaculture ponds (e.g. 310, 311, and/or 312, FIG. 5). In certain embodiments, the harvester is arranged in the predominant wind direction (e.g. FIG. 4) for the area encompassing one, two or more such as the plurality of algal aquaculture ponds as described herein. In this way, algae can be easily harvested.

In one embodiment the system comprises an algal aquaculture pretreatment unit (302, FIG. 5), wherein two or more or all of the components of the algal growth medium 2 (FIG. 1) can be mixed before feeding the algal growth medium 2 to one or more algal aquaculture ponds (e.g. 310, 311, and/or 312, FIG. 5) for growth of algae. In one embodiment, the system comprises one or more filters before the one or more algal aquaculture ponds (e.g. 310, 311, and/or 312, FIG. 5) for growth of algae.

In another aspect, there is provided a use of any embodiment of an algal aquaculture pond 1 or system (e.g. FIG. 5, FIG. 6) as described or encompassed by the current description for cultivating and/or culturing algae.

In certain embodiments, any suitable flow control devices may be utilized for the input and output of material to the algal aquaculture pond. In one embodiment, one or more weir boxes are used for hydraulic flow control of the algal growth medium into and out of the algal aquaculture pond. The weir boxes may be constructed from concrete, wood, high density polyethylene, other materials, or combinations thereof. They may also be fitted with slots to hold screens or barriers to flow. A variety of weirs have been used to control the flow of media in streams, the so-called sharp-crested and trapezoidal forms being relatively common; but broad-crested, triangular, contracted and/or submerged weirs are also favored in certain circumstances. Spillways, controls, and embankments designed to control discharge are simply different kinds of broad-crested weirs. Weirs may be used to control the effluent flow of the algal growth medium from the algal aquaculture pond.

The algal aquaculture pond 1 may further comprise any suitable structures for input and output of materials from the algal aquaculture pond. In an embodiment, when a weir is present, the discharge of the weir is typically through a pipe that is connected to the weir to form a seal. This discharge pipe may be constructed of PVC (polyvinyl chloride), HDPE (high-density polyethylene), cement, or any other suitable piping materials known in the art. The pipe typically runs below the algal aquaculture pond border or dike, and discharges into an adjacent algal aquaculture pond. This discharge from this pipe may be partially diverted to prevent excessive erosion of the bottom of the discharge algal aquaculture pond.

It is further contemplated that the slope of the algal aquaculture pond 1 borders (or dikes), or sides of the algal aquaculture pond, may be important to protect from both erosion and loss of algae due to deposition on the sides from wind and wave action. In certain embodiments, the dike comprises a flat area where vehicular traffic is possible. When looking at a cross-sectional area of a dike, it can comprise two triangular sides that support the center rectangular “crown” on which vehicular traffic is possible. Typical algal aquaculture pond construction will have the triangular side slopes in the range of 1:n, (elevation of the dike above the algal aquaculture pond bottom: distance from the crown of the dike to the algal aquaculture pond bottom) where n is in the range of 3 to 6. A steep slope of 1:1 is preferred because it requires the least amount of earth/rock to be moved into place. However, soil compaction and its ability to resist erosion impacts the exact slope to be used.

Further, depending on the equipment available and the type of soil, a slope of 2:1 might be achievable, for a maximum slope. Too shallow of a slope would result in loss of algal aquaculture pond volume, but the minimum acceptable slope would be about 1:10. The larger the value of n, the more earth that needs to be used to construct the dikes, and thus the higher capital cost of the aquaculture system. If the slopes are too steep, they may erode due to wave action. It is desirable to minimize erosion because that will lead to the need for dike repair. The side slope of the algal aquaculture ponds may be covered with plastic or other material to minimize erosion and to prevent the accumulation of microalgae on the side slope.

In certain embodiments, the bottom of the algal aquaculture pond 1 may be sloped from inlet to outlet in some fashion to allow rapid and complete drainage of the algal aquaculture pond contents. This type of sloping is typically required for shrimp algal aquaculture pond designs, for example, to prevent shrimp from being stranded on the algal aquaculture pond bottom when it is being drained to harvest the shrimp. The minimum slope will be that achievable with available earth-moving equipment. Currently, this is about 5 cm per 1,000 meters (a 0.005% slope). In addition to being sloped from inlet to outlet, the bottom of an algal aquaculture pond may be sloped from side-to-side or to the center, or any cost-effective configuration that allows complete drainage of the algal growth medium from the algal aquaculture pond. More typical sloping is on the range of 2-20 cm per 500 meters.

The algal aquaculture pond 1 may be of any suitable size, including depth, and can be defined or surrounded by one or more borders. The borders may naturally occur, be man made, or a combination thereof. In addition, the algal aquaculture pond 1 may comprise any suitable shape, such as an oval shape. In certain embodiments, the algal aquaculture pond 1 comprises a polygonal shape. In a particular embodiment, the algal aquaculture pond may have a trapezoidal shape. In one embodiment, a cross-section of the algal aquaculture pond is trapezoidal.

As mentioned above, the algal aquaculture pond 1 may be of any suitable shape, such as circular, square, triangular, trapezoidal, or rectangular, but the shape will be designed to conform to any site topographical constraints. In an embodiment, a rectangular shape is employed in a design where the length to width aspect is a function of the number of inlet and/or outlet flow control structures, as well as, the purpose of the particular algal aquaculture pond, i.e., growth or nurturing. For each inlet structure, the portion of the algal aquaculture pond under the influence of a particular inlet will typically have a length to width ratio of at least 2:1 (growth function), with a maximum of about 10:1 (nurturing function). The growth function is where the algae tend to multiply and increase in number. The nurturing function is where the algae are subjected to environmental conditions that result in the expression of desired products. These environmental conditions include, but are not limited to depleted nitrogen fertilizer concentrations, increased solar radiation, increased aquaculture medium temperature, and combinations thereof. The aquaculture system may comprise a multiplicity of algal aquaculture ponds arranged in parallel, series, or a combination thereof. In an embodiment, the algal aquaculture ponds are oriented in series to establish a nutrient concentration profile along the flow path of the algae.

In certain embodiments, there may be provided inlet and outlet hydraulic control structures located on opposite sides of the algal aquaculture pond to minimize short-circuiting. Inlet density differences, along with wind action, may result in decreased residence time of a significant fraction of the influent culture. Optionally, the influent flow through the hydraulic control structure can be baffled in a way to take advantage of the associated energy and provide immediate mixing and dispersion of the culture in that end of the algal aquaculture pond. Baffles may be fitted within the algal aquaculture pond to minimize the potential for short circuiting. The outlet may be fitted with adjustable weirs to allow for the adjustment of algal growth medium height. It may be advantageous to include multiple inlet or outlet control structures in a single algal aquaculture pond.

In addition to water level control via weirs, the algal aquaculture pond 1 may be equipped with an outlet located to permit total drainage of the algal aquaculture ponds.

Depending on the Fetch 3 of the algal aquaculture ponds 1 and the prevailing wind speed 5, there may be sufficient agitation to suspend clay and/or other materials on the bottom the algal aquaculture ponds 1. For example, a wind speed of 2.5 m/see (5.6 mph) can produce a wave mixed layer 7 of 0.58 meters. Thus, if the algal aquaculture pond depth is less than 0.58 meters, some suspension of particles from the bottom layer may occur.

At the outlet of the algal aquaculture pond 1, it may be desirable to have the algal growth medium 2 free of suspended algal aquaculture pond matter. This may be achieved by having a quiescent zone extending about 2 m to about 50 m from the outlet structure(s), e.g., weir, and extending from a nominal algal aquaculture pond depth as a gradient to e.g., about 0.5 to about 2 meters in depth at the outlet. In a particular embodiment, the gradient depth range is from about 0.5 to about 1 meter. Another advantage of the quiescent zone would be to allow phototaxic algae to move to the surface, as the wave mixed layer would decrease.

In an embodiment, it may be desirable to have each algal aquaculture pond 1 accessible from at least two sides by a motorized vehicle, therefore the width of top of any berms (the crown) may be at least about 3 to about 5 meters, or more preferably about 3.3 to about 4 meters. The berm height should be such that adequate freeboard is available to prevent breech by the algal aquaculture pond contents. Wind action can cause waves and seiches, a wave on the surface of a lake or landlocked bay, caused by atmospheric or seismic disturbances that could raise the water level at a berm by six to twelve inches. The minimum freeboard can be one foot. The maximum freeboard can be a function of berm width and the amount of soil required for the berm's construction. Typical berm cross-sections will be trapezoidal so the required minimum width at the top, along with the desired side slope, will set the necessary height. In one embodiment, maximum freeboard is between two and six feet. Any more freeboard can make access to the algal aquaculture pond contents difficult, with the exception of the facility's perimeter berm. Berms for motorized vehicles, including ATVs, can be topped with a layer of road gravel to improve drivability.

As discussed above, a longest dimension each algal aquaculture pond 1 provided may be oriented in the direction of the predominant wind direction for an area at least encompassing the algal aquaculture pond. This will aid in wind mixing the algal growth medium, circulation of the algal growth medium about the algal aquaculture pond, and movement of the grown algal culture to an outlet end of the algal aquaculture pond.

In certain embodiments, the predominant wind direction (e.g. FIG. 4) will be obtained from local wind roses for the area encompassing the algal aquaculture pond. In certain embodiments, when a plurality of algal aquaculture ponds is provided, selected algal aquaculture ponds may be oriented in one direction to orient algal aquaculture pond to the predominant wind during one period of time, and other associated algal aquaculture ponds may be oriented in another direction to exploit winds from another direction during a different period of time.

Fetch 3 (FIG. 1) is the distance of water surface over which the wind blows unimpeded in an essentially constant direction, thus generating waves. The term also is used as a synonym for Fetch length, which is the horizontal distance over which wave-generating winds blow. In one embodiment in an enclosed body of water, Fetch can be defined as the distance between the points of minimum and maximum water-surface elevation. This line generally coincides with the longest axis in the general wind direction. Fetch is an important factor in the development of wind waves.

With respect to Fetch 3 utilized in the present disclosure, it is understood that waves will continue to grow as long as there is a net addition of energy to them. Their height will increase as a function of wind speed and duration and the distance over which it blows (Fetch). Studies in large lakes have shown that the height of the highest waves is related to the Fetch.

Wind blowing over a calm lake surface first produces an effect that may appear as a widely varying and fluctuating ruffling of the surface. The first wave motion to develop is relatively regular, consisting of small, uniformly developed waves called capillary waves. These are quite transient, dissipating rapidly if the wind dies away or developing to the more commonly observed and more persistent gravity waves.

Energy will be continually fed to the waves by the frictional drag of the air moving over the water and by the direct force of the wind on the upwind face of the waves. The latter effect occurs only while the waves move more slowly than the wind. Pressure differences at the air-water interface also contribute energy to surface waves. Energy losses occur due mainly to turbulence in the water and, to a smaller extent, to the effects of viscosity. Turbulence provides mixing in the algal aquaculture ponds.

Wave heights in a given portion of an algal aquaculture pond may vary considerably, due to interactions that suppress some waves and amplify others. Waves travel in the same direction as the wind that generated them and at right angles to their crests. If they meet a solid object rather than a sloping beach, much of their energy will be reflected. If they enter shallow water obliquely, they are refracted.

Aspects of the present disclosure enable the construction and use of algal aquaculture ponds that depend on wind vs. mechanical energy for mixing and movement of the algal growth medium and grown algae within the algal aquaculture pond. In certain embodiments, the algal aquaculture pond is oriented so that the Fetch of the algal aquaculture pond will be in the predominant wind direction. Such a design will allow wind to act as the mixing energy throughout the algal aquaculture pond's water column. The Fetch of the algal aquaculture pond can be the longest dimension subject to unimpeded wind action. Algal aquaculture pond orientation may be based on the local wind rose that will show the prevailing wind for the area encompassing the algal aquaculture pond. Alternatively, if the minimum WML is satisfied by a combination of Fetch and Wind Speed, any algal aquaculture pond orientation is permissible.

Carbon dioxide emissions from fixed sources, such as power plants, have raised considerable controversy. Various techniques have been proposed to capture the carbon dioxide at its source prior to its release into the atmosphere. A significant problem with this approach is that the locations of power plants seldom provide adequate land space or appropriate climate for the growth of algae. The cost of isolating, compressing and transporting carbon dioxide to suitable growth sites will likely be cost prohibitive. A simpler approach is to let nature provide the transportation. By having a highly effective method of isolating carbon dioxide from the air, one can eliminate compression and transportation costs. In accordance with another aspect of the present disclosure, through proper location and alignment of the algal aquaculture ponds with the predominant wind direction (if necessary) as described herein, local wind can provide more than adequate mass transfer of carbon dioxide into the algal growth medium to achieve commercially desirable rates of growth as is illustrated in the analysis below.

The rate of carbon dioxide mass transfer from the atmosphere into an algal growth algal aquaculture pond is impacted by a number of phenomena comprising mass transfer through the gas phase, the gas-liquid interface, the liquid phase, and the liquid film surrounding the algae. Superimposed on diffusional mass transfer are a number of chemical reactions comprising carbonate formation, the uptake of the carbon dioxide by the algae, and the evolution of carbon dioxide by the bacteria. Understanding whether the gas or liquid phase offers the dominant resistance to transport is important to determine whether carbon dioxide transport directly from the atmosphere provides sufficient carbon dioxide to the algae so that carbon dioxide is not the rate-limiting nutrient.

Algae produce oxygen during daylight hours, and they consume oxygen during the night. Furthermore, bacteria in the algal aquaculture consume oxygen during both the day and night and give off carbon dioxide. Thus, the transport of oxygen between the algae-bacteria ecosystem and the atmosphere is critical to the control of an algal aquaculture. Oxygen transport encounters the same diffusional resistances as does carbon dioxide, namely mass transfer through the gas phase, the liquid-gas interface, the liquid phase, and the liquid film surrounding the algae and the bacteria. Superimposed on the diffusional mass transfer are the reaction rate of oxygen evolution or consumption by the algae and oxygen production by the bacteria.

If the gas phase offers the dominant resistance to interfacial mass transfer of oxygen and carbon dioxide, then the expensive step of intimately contacting the oxygen and carbon dioxide gases with the algal aquaculture media is not necessary. Thus, the resistance to diffusional mass transfer through the gas, interface, and liquid are herein examined. Typically, the interfacial resistance to mass transfer is negligible as long as surfactant films have not accumulated at the interface. Furthermore, in gas-liquid transport of carbon dioxide between the atmosphere or flue gas and an aqueous solution, mass transfer through the gas-liquid interface does not dominate the mass flux. Thus, the gas-liquid interfacial mass transfer is neglected in this analysis because it does not offer the dominant resistance to carbon dioxide or oxygen transport into or out of the algal aquaculture ponds.

Previous mass transfer calculations for carbon dioxide transport into an algal growth algal aquaculture pond showed that the liquid-phase mass transfer resistance dominated the mass transfer rate [see Putt-Algae as a Biodiesel Feedstock: A Feasibility Study, Appendix A: Air-to-Algal aquaculture pond Carbon Dioxide Transport, submitted to NREL (2008)]. This analysis was based on the mass transfer model by Wanninkhof (1992). To approximate the mass transfer of carbon dioxide into the algae algal aquaculture pond, Putt assumed the wind speed to be 5 cm/hr. This translates to a mean wind speed of 0.0000139 m/s (or 0.00003 miles per hour). To provide an even more conservative analysis, the gas phase transport was taken when the wind speed approaches zero. While this analysis appears to be very conservative, resulting in the smallest transport and lowest rate, a better approximation is to use the model for low rates of mass transfer developed for conservative analysis of environmental releases by the U.S. EPA (Guidelines (2000). This method, using the Pasquill-Gifford model, results in the worst-case mass transfer circumstances corresalgal aquaculture ponding to Class F conditions with a wind speed of 1.5 m/s or Class E conditions and 2.0 m/s, both values significantly higher than the previous assumption. It is these two conditions that are used for detailed environmental release calculations by the U.S. EPA and is thus the wind speed used for carbon dioxide and oxygen mass transfer rate predictions herein. Furthermore, the analysis of Putt completely neglects the impact of mass transfer in combination with chemical reaction, as was previously discussed. The addition of these parameters will show that the gas phase mass transfer resistance dominates the liquid phase mass transfer resistance. Thus, the carbon dioxide and oxygen transport will be dominated by the gas phase mass transfer coefficient.

Carbon dioxide transport into liquids has been widely studied. Both Treybal (1980) and Skelland (1974) have examples that discuss the transport of carbon dioxide into water. Treybal calculates an effective transport film thickness for transport of carbon dioxide from a pure carbon dioxide gas phase into water of approximately 240 microns. The absorption of carbon dioxide from industrial process streams has also been evaluated for many years. In order to enhance the rate of mass transfer, many absorption process liquids have been used including carbonate and ethanolamine aqueous solutions, specifically for the absorption of carbon dioxide from flue gas.

The analysis of the effect of having a reaction in the liquid phase has been analyzed theoretically and measured experimentally. Rochelle (2006) concluded, based on experimental data, that the gas phase offered the dominant resistance to carbon dioxide transport into an amine solution, and it contributed more than 80% of the resistance to mass transfer of the carbon dioxide into the aqueous solution. The analysis using a combination of reaction and mass transfer is what more accurately describes the carbon dioxide transport into algae growth algal aquaculture ponds. The effect of the algae is to rapidly consume any carbon dioxide in the liquid phase as a food source. Studies of absorption with chemical reaction are described in Dankwerts (1970), Sherwood (1975), and Astarita (1983), and many other references including the classic paper of Kohl (1979).

Carbon dioxide removal from a dilute process gas stream can be enhanced by adding a chemical reaction in the liquid phase. The net effect is to reduce the resistance to mass transfer in the liquid to nearly zero when compared to the resistance in the gas phase. Dankwerts (1970) offers many examples of carbon dioxide transport into water and into a solution with rapid chemical reaction. Dankwerts calculated the enhancement factor that applies for carbon dioxide to be approximately 44. This means that the mass transfer rate in the liquid phase is 44 times the rate without reaction. To compare with analyses that do not consider reactions, such as those of Putt (2008), the effective liquid-side film thickness for mass transfer needs to be reduced by a factor of 44. The net effective rate of transfer in the liquid will be enhanced by this factor.

For Putt's analysis the carbon dioxide transport on the liquid side will be approximately 0.35×44=15.4 grams of carbon dioxide per square meter per day, or 15.4 g CO₂/m²/day, which is significantly higher than Putt's calculated rate for the gas side mass transfer of 0.84 g CO₂/m²/day, even with the unrealistically low velocity that was used. Taking into account the effect of liquid side reaction, the ratio of mass transfer on the liquid side to gas side is approximately 18. Dankwerts discusses the relative rate of reaction to mass transfer for gas-liquid systems. Carbon dioxide is only used as an example of a rapid reaction system. Sherwood et al. has an entire chapter on mass transfer with reaction and presents several graphs of the rate of mass transfer of carbon dioxide into liquids. Sherwood et al. conclude in FIGS. 8.26 through 8.28 on pages 368-369, that the systems with carbon dioxide have rapid bimolecular reactions and depend on the rate of reacting species rather than on the linear rate of mass transfer of carbon dioxide. The effect of this analysis is to have the rate of mass transfer in the algal aquaculture ponds to be dominated by the rate that algae can consume the carbon dioxide.

Astarita (1983) further states on page 144 that “CO₂ mass transfer very often takes place in the fast reaction regime.” Astarita provides a survey of alternative absorption solvents for recovering it from industrial process streams. Thus, it is the gas phase mass transfer rate that needs to be calculated to determine if mass transfer is limiting algal growth in large open algal aquaculture ponds. Furthermore, since the gas phase dominates the mass transfer resistance to carbon dioxide, and by analogy the oxygen transport to and from algal aquaculture ponds, the rate of gas transport must be understood in order to determine if the oxygen and carbon dioxide mass transfer is controlling the algal growth.

The rate model used for predicting the gas phase mass transfer assumes that the transport in the gas phase is near the leading edge of a flat plate. The mass transfer predicted will be lower than that for flow far from the leading edge. Thus, the mass transfer coefficient predicted will be lower than that obtained across algal aquaculture ponds greater than one (“1”) meter in width.

The mass transfer models used to predict oxygen and carbon dioxide transport both to and from the algal aquaculture ponds from the atmosphere are founded in transport equations from Treybal (1980), the contents of which are included herein by reference.

In evaluation of the transport of oxygen and carbon dioxide to the algal growth algal aquaculture ponds, the effect of the reactions with the algae and carbonate formation reduces the liquid-side mass transfer resistance to zero, based on previous arguments. The ratio of gas-phase mass transfer to liquid-phase mass transfer with rapid reaction in the liquid phase is dominated by the lowest rate which is in the gas phase. The carbon dioxide concentration in the liquid is reduced from its low equilibrium liquid solubility by the algae that use the carbon dioxide as a carbon food source. One might consider the ratio of mass transfer coefficients for carbon dioxide transport into a liquid amine solution as an analogous situation. In that case, the mass transfer coefficient in the liquid phase is at least an order of magnitude larger than the gas-side mass transfer coefficient. Thus, the mass transfer is controlled by the lowest transport rate, i.e. the carbon dioxide transport in the gas phase. The ratio of the individual phase mass transfer coefficients, -kx/ky, wherein the value of the liquid phase coefficient (kx) is divided by that for the gas phase coefficient (ky), reflects the relative ratio of transport resistance in the liquid phase to the transport in the vapor phase.

For carbon dioxide transport with reaction in the liquid phase, this ratio approaches infinity effectively reducing the liquid phase carbon dioxide concentration to near zero. The gas-side mass transfer is approximated by transport over a flat plate. Because of wind effects, transport is approximated by a short distance flat plate. The American Institute of Chemical Engineers, AIChE, Design Institute for Multiphase Flow (DIMP) project evaluated fluid flow for vapor-liquid interfaces and verified mass transfer relationships when the flow was between a vapor phase (such as the atmosphere) over a liquid phase (such as an algae algal aquaculture pond). According to the U.S. EPA, the basic fluid mechanics that apply for the atmosphere is equivalent to a ventilation rate of approximately 85 cubic meters per minute (3000 ft³/min). This translates to average flow conditions having linear velocities between 1.0 and 2.0 m/s, which provides turbulent flow in the gas phase. When the wind is in direct contact with an open algal growth algal aquaculture pond, as the turbulent flow increases, it generates two- and three-dimensional (2-D and 3-D) waves.

The turbulent mechanism calls for the vapor flow to be connected to the liquid surface for a short distance and then be replaced with new vapor phase flow. This periodic surface renewal must be considered when calculating an effective transport distance for mass transfer over a flat plate. In this case, one meter is used as a short distance to use. If a longer distance is used, a higher mass transfer rate results. The basic mass transport mechanism assumes a boundary layer flow over a flat plate as a conservative prediction of the mass transfer. Limited flow over a short flat plate is assumed, and as the rate of mass transfer is augmented a higher rate of mass transfer is realized. Because of turbulence, small waves will usually form and appear as ripples. This effect will increase the rate of mass transfer; however, it is not considered in this analysis. The atmospheric conditions across an algal aquaculture pond are estimated to corresalgal aquaculture pond to conservative atmospheric conditions for a neutral buoyancy model, Stability Class F, wind speed of 1.5 to 2 m/s (3.4 to 4.5 miles per hour), see for example Crowl (2002).

The use of these models is based on the design of the algal growth algal aquaculture ponds. The algal growth algal aquaculture ponds have a surface that has no frictional effects that will increase a ground roughness and reduce the velocity needed to predict the rate of mass transfer. An expected surface roughness (z0) over the algal aquaculture pond is negligible and is on the order of 10⁻⁴. (See Guidelines for Chemical Process Quantitative Risk Analysis, 2^{nd}. ed. Center for Chemical Process Safety, American Institute of Chemical Engineers, New York (2000).

$$L = \frac{g {T}^{2}}{2 π} \qquad Equation ( 6 )$$

$$T = \frac{2.4 πU tanh [ 0.077 {( \frac{g F}{{U}^{2}} )}^{0.25} ]}{g} \qquad Equation ( 7 )$$

$$WML = \frac{L}{2} \qquad Equation ( 8 )$$

$$\begin{matrix}Where : \\ U = wind speed , m {sec}^{- 1} \\ F = Fetch , m \\ L = wavelength , m \\ T = wave period , sec \\ g = gravitational constant , {9.8 m sec}^{- 2} \\ WML = wave mixed layer , m\end{matrix}$$

Bottom sediments of earthen lined algal aquaculture ponds with a long Fetch are subject to resuspension in the water column depending on the strength (speed) and duration of the prevailing winds. Additionally, this effect will depend on the depth of the algal aquaculture pond and the wave mixed layer generated by the wind. The type of bottom sediment will also play a role in how much sediment might be resuspended. Generally speaking, the potential for resuspension begins when the depth of the algal aquaculture pond is less than one-half of the wavelength. As seen in Equation 6, the wavelength, L, is a function of the wave period, T. The wave period can be estimated using Equation 7 that shows how it is related to both Fetch, F, and wind speed, U. This empirical equation was developed by the US Army Coastal Engineering Research Center (Carper and Bachmann, 1984) and is used in the following example for determining the depth of mixing in the algal aquaculture ponds.

In one embodiment, the ratio of the WML to the pond depth (e.g. a total depth of the algal growth medium in the algal aquaculture pond) is selected so that the desired mixing of the nutrients is achieved but the sediment is not suspended to cause turbidity of the algal aquaculture pond.

In one embodiment, the algal aquaculture pond is designed with a Fetch so that the ratio of the WML to the pond depth (e.g. a total depth of the algal growth medium in the algal aquaculture pond) is greater than 0.2, 0.3, 0.4, 0.5, 0.6, 0.7, 0.8, 0.9, 1, 1.5, or 2 so that the desired CO₂ and/or O₂ mass transfer is achieved.

In one embodiment, the algal aquaculture pond is designed with sufficient Fetch so that CO₂ and/or O₂ gas phase mass transfer does not limit algal growth.

In one embodiment, the algal aquaculture pond is designed with a Fetch so that the CO₂ and/or the O₂ gas phase mass transfer coefficients are greater than about 1, 5, 7, 9, 10, 11, 12, 13, 14, 15, 16, 17, 18, 19, or 20 kmol/m²/day.

A wind blowing across the surface of a water body, creating wave action will mix the underlying water layer to a depth defined as the wave mixed layer, WML. This depth will be a function of the wave length or the distance, L, measured from crest to crest of the waves. This distance is a function of the wave period, T, which is the time between wave crests to pass a given point. The strength (speed) of the prevailing wind, U, influences both the wave length, L, and wave period, T. For the wind to generate waves, it must have a minimum distance of unimpeded contact with the surface of the algal aquaculture pond. This distance is called the Fetch, F.

Table 1 presents the wave properties for a shallow algal aquaculture pond with a Fetch of one kilometer and winds ranging from 1.1 mph to 11.2 mph. If the average depth of the algal aquaculture pond is 0.5 m then the mixing depth, WML, due to the wind will have the potential to resuspend bottom sediment at wind speeds of greater than approximately 5 mph.

In certain aspects of the present disclosure, the following components and/or parameters may be considered and incorporated in an algal aquaculture pond, system, or process as described herein.

Freeboard—The distance between normal water level and the top of an embankment, that impounds or restrains water.

Side Slope—The slope of the sides of the algal aquaculture pond will be important to protect from both erosion and loss of algae due to deposition on the sides from wind and wave action. Typical algal aquaculture pond construction will have side slopes in the range of 1:n, where n is in the range of 3 to 6. However, a slope of 1:1 is preferred, but depending on the equipment available, a slope of 2:1 might be achievable, for a maximum slope. Too shallow would result in loss of algal aquaculture pond volume, but the minimum acceptable slope would be 1:10. The Angle of Repose for the material should not be exceeded to avoid unstable construction.

Angle of Repose—The Angle of Repose is an engineering property of granular materials. The angle of repose is the maximum angle of a stable slope determined by friction, cohesion and the shapes of the particles.

When bulk granular materials are poured onto a horizontal surface, a conical pile will form. The internal angle between the surface of the pile and the horizontal surface is known as the angle of repose.

Bottom Slope—The bottom of the algal aquaculture pond may be sloped in some fashion to allow rapid and complete drainage of the algal aquaculture pond contents. This ability is a necessary operational maneuver in the control of various predators and competitors in the growth sequence of the desired algal species. This minimum slope is achievable with the available earth-moving equipment. The bottom can be sloped from inlet to outlet, or side-to-side, or to the center of the algal aquaculture pond, or any cost effective configuration that allows complete drainage.

Shape, Length and Width-Algal aquaculture ponds can be any suitable shape, such as circular, square, triangular, trapezoidal, or rectangular, but the shape will be designed to conform to any site topographical constraints. Typically a rectangular shape will be employed in a design where the length to width aspect will be a function of the number of inlet and/or outlet flow control structures, as well as, the purpose of the particular algal aquaculture pond, i.e. growth or nurturing. For each inlet structure the portion of the algal aquaculture pond under the influence of a particular inlet will have a length to width ratio of at least 2:1 (growth function), with a maximum of 10:1 (nurturing function).

TABLE 1
Predicted wave properties as a function
of wind speed for a 1,000 m Fetch.

| U, m sec−1 | U, mph | T, sec | L, m | WML, m |
|---|---|---|---|---|
| 0.5 | 1.1 | 0.31 | 0.15 | 0.07 |
| 1 | 2.2 | 0.50 | 0.38 | 0.19 |
| 1.5 | 3.4 | 0.64 | 0.64 | 0.32 |
| 2 | 4.5 | 0.76 | 0.90 | 0.45 |
| 2.5 | 5.6 | 0.87 | 1.17 | 0.58 |
| 3 | 6.7 | 0.96 | 1.43 | 0.72 |
| 3.5 | 7.8 | 1.04 | 1.70 | 0.85 |
| 4 | 8.9 | 1.12 | 1.97 | 0.99 |
| 4.5 | 10.1 | 1.20 | 2.24 | 1.12 |
| 5 | 11.2 | 1.27 | 2.51 | 1.26 |

U = wind speed, m sec⁻¹
F = Fetch, m
L = wavelength, m
T = wave period, sec
g = gravitational constant, 9.8 m sec⁻²
WML = wave mixed layer, m

Suspended Particles Settlement Zone-Depending on the Fetch of the algal aquaculture ponds and the prevailing wind speed, there may be sufficient agitation to suspend clay and/or other materials on the bottom of the algal aquaculture ponds. For example, in Table 1 below we see that a wind speed of 2.5 m/sec (5.6 mph) can produce a wave mixed layer of 0.58 meters. Thus, if the algal aquaculture pond is less than 0.58 meters, some suspension of the bottom layer may occur.

In some embodiments, an exit weir may be provided at an outlet of the algal aquaculture pond. At the exit weir, it is typically desirable to have the aqueous growth medium free of such suspended algal aquaculture pond matter. This may be achieved by having a quiescent zone extending about 2 m to about 50 m from the weir and extending from the nominal algal aquaculture pond depth as a gradient to about 0.5 to about 2 meters in depth at the weir. An even more desirable gradient depth range is from about 0.5 to about 1 meter.

Algal aquaculture pond Cross-Section—The cross-section of algal aquaculture ponds can assume a variety of geometries, including a cross-section in which a flat bottom has a side to side slope. Key factors in cross section selection include ease of construction, ease of algal aquaculture pond drainage, and mixing dynamics from Fetch.

Inlet and outlet-When present, inlet and outlet hydraulic control structures may be located on opposite sides of an algal aquaculture pond to minimize short-circuiting. Inlet density differences, along with wind action, may result in decreased residence time of a significant fraction of the influent culture. Optionally, the influent flow through the hydraulic control structure can be baffled in a way to take advantage of the associated energy and provide immediate mixing and dispersion of the culture in that end of the algal aquaculture pond.

Berm Width and Height—In certain embodiments, it may be desirable to have each algal aquaculture pond accessible from at least two sides by a motorized vehicle, therefore the width of top of these berms may be a minimum of about 2 to about 4 meters, and more preferably about 3 to about 4 meters in certain embodiments. The berm height should be such that adequate freeboard is available to prevent breech by the algal aquaculture pond contents. Wind action can cause waves and seiches that could raise the water level at a berm by about 0.1 to 0.2 meters, so the minimum freeboard is 0.3 meters. The maximum freeboard will be a function of berm width and the amount of soil required for the berm's construction. Typical berm cross-section will be trapezoidal so the required minimum width at the top, along with the desired side slope, will set the necessary height. Maximum freeboard would be between about 0.6 to 2 meters. Any more freeboard makes access to the algal aquaculture pond contents difficult, with the exception of the facility's perimeter berm. Berms for motorized vehicles can be topped with a layer of road gravel to improve drivability.

Algal aquaculture pond Orientation—As discussed above, the algal aquaculture pond may be oriented in the direction of the prevailing winds. This will aid in wind mixing and movement of the algal culture to the outlet end of the algal aquaculture pond. The predominant wind direction may be obtained from local wind roses for the site of the algal aquaculture pond. Sometimes the predominant wind direction data will show two equally prevalent wind directions generally in the same direction. In these cases, the Fetch may be the bisect of the two directions and the outlet end of the algal aquaculture pond can be constructed in a V-shape such that an edge of the algal aquaculture pond is perpendicular to the wind direction of each of the prevailing winds. Even if there is a distinct predominant wind direction, the outlet end of the algal aquaculture pond can be constructed in a V-shape to facilitate the efficient capture of the algae in either the algal growth algal aquaculture ponds or the nurturing algal aquaculture ponds.

The algal growth algal aquaculture pond may be of a shape that conforms to the available topography of a chosen site. Common wall or common berm (dike) construction will be utilized wherever possible to reduce the cost of algal aquaculture pond construction. Flow into the algal growth algal aquaculture pond from suitable sources of the algal growth medium, e.g., a pretreatment unit, may be controlled with any one of several types of weir structures, e.g., a submerged weir. A submerged weir can be designed to control the flow into individual algal growth algal aquaculture ponds at a rate that prevents hydraulic surges through the algal aquaculture pond. The layout of the algal aquaculture ponds will be in parallel and may be oriented with the longest dimension thereof in the direction of the Fetch associated with the prevailing winds as defined by the local wind rose. Alternating berms will be of a width adequate for motor vehicle conveyance to afford access to at least three sides of any one of the algal growth bioreactors.

In certain embodiments, a plurality of algal growth algal aquaculture ponds may be provided and the algal growth algal aquaculture ponds may be operated in parallel with each individual algal aquaculture pond receiving a flow adequate to provide a target hydraulic residence time for that particular algal aquaculture pond. The influent flow to all the algal aquaculture ponds may contain the same nutrient concentration, algal biomass seed concentration, and the same salinity, pH, and temperature. Any additional supplements to an individual algal aquaculture pond can be made directly to an algal aquaculture pond using a mobile tank truck for delivery of the supplement.

In certain embodiments, the effluent from the algal aquaculture pond may be split in order to send some algal biomass back to the algal growth algal aquaculture pond headworks, where headworks are any structure or structures at the head or diversion point of a waterway, by way of a preliminary algal growth reactor. In addition, the other portion of the discharge may be directed into any subsequent algal aquaculture pond for the purpose of nurturing, harvesting, or nutrient recovery. In certain embodiments, the outlet of the algal growth algal aquaculture pond may also have a submerged weir for removing growth media containing primarily excess bacteria at times when the bulk of the algal growth is at or near the surface of the algal growth algal aquaculture pond at the outlet end. This may be performed on an as needed basis as defined by target oxidation-reduction potentials measured in the algal growth bioreactors in question.

FIG. 5 and FIG. 6 herein show exemplary systems in accordance with the present disclosure comprising multiple algal aquaculture ponds as described herein. It is understood that each of the components in the illustrated systems are optional and not necessary required. As shown in FIG. 5, an aqueous medium 301 will enter an Algal Aquaculture Pretreatment Reactor 302 where it will be distributed (Line 303) in a flow-controlled manner, using submerged weirs 307, 308, 309 or any other appropriate weir control device, to algal growth algal aquaculture ponds 310, 311, and/or 312. Either prior to the flow control device or post flow control device, the distributed flow may pass through an optional third filtration step 304, 305, 306.

The algal growth algal aquaculture ponds 310, 311, 312 may be of a shape that conforms to the available topography of a chosen site. Common wall or common berm (dike) construction will be utilized wherever possible to reduce the cost of algal aquaculture pond construction. Flow into the algal growth algal aquaculture ponds 310, 311, 312 from an optional algal aquaculture pretreatment reactor 302 may be controlled with any one of several types of weir structures, such as a submerged weir. A submerged weir can be designed to control the flow into individual algal growth algal aquaculture ponds 310, 311, 312 at a rate that prevents hydraulic surges through the algal aquaculture pond. The layout of the algal aquaculture ponds may be in parallel and oriented with the longest dimension in the direction of the Fetch associated with the predominant wind direction as determined by a local wind rose, for example. Alternating berms may be provided with a width adequate for motor vehicle conveyance to afford access to at least three sides of any one of the algal growth algal aquaculture ponds 310, 311, 312.

The algal growth algal aquaculture ponds 310, 311, 312 may be operated in parallel with each individual algal aquaculture pond receiving a flow adequate to provide a target hydraulic residence time for that particular algal aquaculture pond. The influent flow to all the algal aquaculture ponds will contain the same nutrient concentration, algal biomass seed concentration, and the same salinity, pH, and temperature. Any additional supplements to an individual algal aquaculture pond may be made directly to an algal aquaculture pond using a mobile tank truck for delivery of the supplement.

The effluent from the individual algal growth algal aquaculture ponds 310, 311, 312 may be split in order to send some algal biomass back to algal growth algal aquaculture pond headworks by way of the algal aquaculture pretreatment reactor 302. The bulk of the discharge may be directed into any subsequent algal aquaculture pond for the purpose of nurturing, harvesting, or nutrient recovery. The outlet of the algal growth algal aquaculture ponds 310, 311, 312 may also have a weir to remove growth media on a continuous basis. A submerged weir may be used for removing growth media containing primarily excess bacteria at times when the bulk of the algal growth is at or near the surface of the algal growth bioreactor at the outlet end. This may be performed on an as needed basis as defined by target oxidation-reduction potentials measured in the algal growth bioreactor in question.

In another embodiment, as shown in FIG. 6, there is shown a system with a plurality of algal aquaculture ponds in series. In the embodiment, the aqueous medium 401 may enter an Algal Aquaculture Pretreatment Unit 402 where it will be distributed (Line 403) in a flow controlled manner, using submerged weirs 407, 408, 409 or any other appropriate flow control device, to algal growth algal aquaculture ponds 410, 411, and/or 412. Either prior to the flow control device or downstream thereof, the distributed flow may pass through the optional third filtration step 404, 405, 406.

The algal growth algal aquaculture ponds 410, 411, 412 may be of a shape that conforms to the available topography of a chosen site. Common wall or common berm (dike) construction will be utilized wherever possible to reduce the cost of algal aquaculture pond construction. Flow into the algal growth algal aquaculture ponds 410, 411, 412 from the optional algal aquaculture pretreatment reactor 402 may be controlled with any one of several types of weir structures, such as a submerged weir. A submerged weir can be designed to control the flow into individual algal growth algal aquaculture ponds 410, 411, 412 at a rate that prevents hydraulic surges through the algal aquaculture pond. The layout of the algal aquaculture ponds will be in series and wherever possible oriented with the longest dimension of the algal aquaculture ponds in the direction of the Fetch associated with the predominant wind direction as determined by a local wind rose, for example. Alternating berms may be provided a width adequate for motor vehicle conveyance to afford access to at least three sides of any one of the algal growth algal aquaculture ponds 410, 411, 412.

The algal growth/algal aquaculture ponds 410, 411, 412 will be operated in series with each individual algal aquaculture pond receiving a flow adequate to provide a target hydraulic residence time for that particular algal aquaculture pond. The influent flow to all the algal aquaculture ponds will contain the same nutrient concentration, algal biomass seed concentration, and the same salinity, pH, and temperature. Any additional supplements to an individual algal aquaculture pond may be made directly to an algal aquaculture pond using a mobile tank truck for delivery of the supplement.

The effluent from the individual algal growth algal aquaculture ponds 410, 411, 412 may be split in order to send some algal biomass back to the algal growth algal aquaculture pond headworks by way of the algal aquaculture pretreatment unit 402. The remainder of the discharge may be directed into any subsequent algal aquaculture pond for the purpose of nurturing 416, 417, 418, harvesting 420, or nutrient recovery 423. The outlet of the algal growth algal aquaculture ponds 410, 411, 412 may also have a weir that may be submerged for removing growth media containing primarily excess bacteria at times when the bulk of the algal growth is at or near the surface of the algal growth algal aquaculture pond at the outlet end. This may be performed on an as needed basis as defined by target oxidation-reduction potentials measured in the algal growth bioreactor in question.

In an alternative mode of operation, the outflow of each individual algal growth algal aquaculture pond may be directed to an algal aquaculture pond or multiplicity of algal aquaculture ponds in series with each individual algal aquaculture pond. The purpose of these algal aquaculture ponds is to optimize the final stages of growth in a nurturing environment, and therefore these algal aquaculture ponds are known as Nurturing Algal aquaculture ponds shown as 416a,b,c and 417a,b,c, and 418a,b,c in FIG. 6. When the target levels of product are achieved in the nurturing algal aquaculture ponds, the effluent will be sent either directly to the harvester 420, or optionally the algal culture may be pre-concentrated in a pre-concentration step 419. The purpose of the nurturing algal aquaculture ponds 416, 417, 418 is different from the algal growth algal aquaculture ponds 410, 411, 412. Nurturing allows the expression of a desired product, while the algal growth algal aquaculture ponds are for multiplying and increasing the algal biomass.

In both parallel and series modes of operation, the water that is separated from the algal biomass in the harvester 420 may be recycled to a nutrient recovery facility 423 by way of line 418. The purpose of this facility 423 is to recover some of the nutrients in the residual algal biomass through microbial digestion.

Release of macronutrients nitrogen and phosphorus, as well as micronutrients and other trace elements reconditions the algal growth medium and reduces the amount of nutrient supplementation in the algal aquaculture pretreatment reactor 402. When reconditioned in the nutrient recovery facility 423, the algal growth medium is returned to the inlet of the algal aquaculture pretreatment reactor 402 via line 424. The dewatered algal biomass leaves the harvester 420 by line 421. Alternatively, the algal growth medium separated from the algal biomass may be returned to its original source by line 422.

In an exemplary algal aquaculture pond 1 for cultivating algae of the present disclosure, there is the algal aquaculture pond 1 comprising: an algal growth medium 2 optionally comprising added algal nutrients within the algal aquaculture pond 1; and a Fetch 3 capable of mixing the algal growth medium 2 and/or circulating the algal growth medium 2 throughout the algal aquaculture pond 1, wherein the ratio of a wave mixed layer 7 of the algal growth medium 2 to a total depth 9 of the algal growth medium 2 in the algal aquaculture pond 1 is greater than about 0.2.

In an exemplary algal aquaculture pond 1 as disclosed herein, the algal aquaculture pond is a manmade algal aquaculture pond.

In yet another exemplary embodiment there is provided the algal aquaculture pond 1, wherein at least a portion of the algal nutrients are intentionally added to an aqueous stream to form the algal growth medium 2.

In an exemplary algal aquaculture pond 1 as described herein, there is provided the algal aquaculture pond 1 wherein a flow of the algal growth medium 2 out of the algal aquaculture pond 1 is continuous.

An exemplary algal aquaculture pond 1 wherein the depth (i.e. total depth or for example the depth from the surface of the pond for the WML) of the algal growth medium in the algal aquaculture pond 1 is a mean depth of the algal growth medium 2 in the algal aquaculture pond 1, optionally based on the depth throughout the entire algal aquaculture pond area, is disclosed.

Another exemplary algal aquaculture pond 1 as disclosed herein, wherein the ratio of a wave mixed layer 7 of the algal growth medium 2 to a depth of the algal growth medium 2 in the algal aquaculture pond 1 is selected to be greater than about 0.3, greater than about 0.4, greater than about 0.5, greater than about 0.6, greater than about 0.7, greater than about 0.8, greater than about 0.9, and/or greater than about 1.0.

In an exemplary embodiment is the algal aquaculture pond 1 wherein the Fetch 3 is a distance from about 75 to about 3000 meters, from about 80 to about 2000 meters, or from about 100 to about 1500 meters, optionally across the longest dimension of the algal aquaculture pond 1.

In another exemplary embodiment there is provided the algal aquaculture pond 1 according to the present disclosure, wherein the algal aquaculture pond 1 comprises a bottom slope from an inlet to an outlet of the algal aquaculture pond 1, and optionally said bottom slope is at least about 5 cm per 1000 meters of length.

In a further exemplary embodiment, there is provided the algal aquaculture pond 1 as disclosed herein, wherein the depth of the algal growth medium 2 is from about 0.15 to about 2 meters.

An exemplary algal aquaculture pond 1 wherein the depth of the algal growth medium is less than the wave mixed layer 7 to ensure mixing of the algal growth medium 2 or mixing the aqueous medium with the algal nutrients and/or to ensure circulation of the algal growth medium 2 throughout the algal aquaculture pond 1, is also disclosed.

Another exemplary algal aquaculture pond 1, wherein a longest dimension of the algal aquaculture pond 1 is arranged in a predominant wind direction for an area encompassing the algal aquaculture pond 1, optionally determined from historical wind direction data. For example, in the directions shown in FIG. 4.

An exemplary algal aquaculture pond 1 is disclosed herein, wherein the longest dimension of the algal aquaculture pond 1 is or is arranged within about 30 degrees, about 25 degrees, about 20 degrees, about 15 degrees, about 10 degrees, or about 5 degrees of the predominant wind direction, optionally determined from wind direction data. For example, in the wind directions shown in FIG. 4.

Disclosed herein is the algal aquaculture pond 1, wherein the wind direction data comprises wind rose data for the area encompassing the algal aquaculture pond 1 for a duration of selected to be at least about 6 months, at least about 1 year, at least about 2 years, at least about 5 years, and/or at least about 10 years.

An exemplary algal aquaculture pond, wherein the predominant wind direction represents a predominant wind direction during daylight hours, is also disclosed herein.

There is also provided an exemplary algal aquaculture pond 1 as described herein, wherein a cross-section of the algal aquaculture pond 1 is trapezoidal.

An exemplary algal aquaculture pond 1 as disclosed herein, wherein the algal aquaculture pond 1 comprises an exit-weir for overflow of the algal growth medium 2 and/or comprises a drain to permit at least partial withdrawal or complete withdrawal of the algal growth medium 2 from the algal aquaculture pond 1.

An exemplary algal aquaculture pond 1 is disclosed, wherein the wave mixed layer 7 of the algal growth medium 2 in the algal aquaculture pond 1 is greater than about 1.5 cm or greater than about 2.5 cm.

An exemplary algal aquaculture pond 1 of the present disclosure, wherein the wave mixed layer 7 of the algal growth medium is calculated according to the equation:

$$WML = WML = {e}^{[ - 0.18691 + 0.48936 * Ln ( Fetch ) + 1.0365 * Ln ( Wind Speed ) ]} ; wherein e = 2.7 \; 182818284 and WML$$

is the wave mixed layer.

An algal aquaculture pond as described in an exemplary embodiment herein, wherein the algal aquaculture pond 1 further comprises a quiescent zone optionally at an end of the algal aquaculture pond, such as at the exit-weir end of the algal aquaculture pond.

An exemplary algal aquaculture pond 1, wherein the quiescent zone comprises a greater depth than an adjacent portion of the algal aquaculture pond, is also disclosed.

There is provided an exemplary algal aquaculture pond 1, wherein the algal aquaculture pond is capable of producing and maintaining an algal concentration of about 20,000 to about 2,000,000 algal cells per milliliter averaged over about a top 15-30 cm of a depth of the algal growth medium or over about a top 10% of a depth of the algal growth medium.

Also provided is an exemplary algal aquaculture pond 1, wherein dissolved inorganic carbon or a mass transfer of carbon dioxide in the algal aquaculture pond satisfies an algal concentration of about 20,000 to about 2,000,000 cells per milliliter averaged over about a top 15-30 cm of a depth of the algal growth medium or over about a top 10% of a depth of the algal growth medium.

Another exemplary algal aquaculture pond 1 is provided, wherein a salinity of the algal growth medium is at least about 7 wt %, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt %.

An algal aquaculture pond 1 in yet another exemplary embodiment is provided, wherein the algal aquaculture pond comprises algae, and wherein the algae are selected from microalgae; phototaxic microalgae; one or more of the following algae: Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., and genetically-engineered varieties or combinations (mixtures, or mixed cultures) thereof; and one or more of the following microalgal species including Amphora sp., Ankistrodesmus, Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schiochytrium limacinum, Stichococcus sp., Tetraselmis suecica, and Thalassiosira pseudonana, and genetically-engineered varieties or combinations (mixtures, or mixed cultures) thereof.

An exemplary algal aquaculture pond 1 is disclosed, wherein the algal aquaculture pond does not comprise one or more mechanical mixing device(s).

An exemplary system for cultivating algae is provided, wherein the system comprises one, two or a plurality of the algal aquaculture ponds described herein.

An exemplary system, wherein a longest dimension of one algal aquaculture pond, two algal aquaculture ponds or each of the algal aquaculture ponds is arranged in the predominant wind direction, is disclosed.

An exemplary algal aquaculture pond 1 or system of the present disclosure is provided, further comprising a harvester for harvesting algae in fluid communication with the algal aquaculture pond or the algal aquaculture ponds, wherein the harvester is also optionally arranged in the predominant wind direction for the area encompassing the algal aquaculture pond or algal aquaculture ponds.

The use of the algal aquaculture pond 1 or system of the present disclosure is provided for cultivating algae.

An exemplary process for cultivating algae, wherein the process comprises cultivating algae in the algal aquaculture pond or the system of the present disclosure, and optionally harvesting the cultivated algae.

An exemplary process is provided herein, wherein the process comprises determining a predominant wind direction from wind direction data for a predetermined area; and arranging a longest dimension of the algal aquaculture pond or one, two, or each of the algal aquaculture ponds in the determined predominant wind direction within the predetermined area.

An exemplary process, further comprising constructing the algal aquaculture pond based on historical wind direction data for an area encompassing the algal aquaculture pond to provide an amount of Fetch capable of growing algae in the algal aquaculture pond.

An exemplary process is disclosed, wherein the process comprises providing the algal aquaculture pond 1 with an algal growth medium for the growth of the algae in the algal aquaculture pond, the algal growth medium comprising an aqueous medium and algal nutrients.

An exemplary process for growing algae is disclosed herein comprising: determining a predominant wind direction from historical wind direction data for a predetermined area; based on the predominant wind direction, providing an algal aquaculture pond with: (i) a Fetch capable of mixing an algal growth medium in the algal aquaculture pond and/or circulating the algal growth medium throughout the algal aquaculture pond, and (ii) a ratio of a wave mixed layer of the algal growth medium to a depth of the algal growth medium in the algal aquaculture pond of greater than about 0.2; and growing algae in the algal aquaculture pond.

An exemplary process is disclosed herein, wherein the providing of the algal aquaculture pond 1 comprises constructing the algal aquaculture pond such that a longest dimension of the algal aquaculture pond is arranged in the determined predominant wind direction for the predetermined area.

Another exemplary process is disclosed, wherein a salinity of the algal growth medium 2 is at least about 7 wt %, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt %.

### EXAMPLES

### Example 1: Fetch Mixing of Minerals in an Algal Aquaculture Pond

An algal aquaculture pond 1 with a Fetch 3 length of about 1,400 meters was sampled weekly for three weeks to determine how the mineral content would vary over time.

The dominant resistance to mass transfer of oxygen out of algal aquaculture ponds is assumed to be in the gas phase. Resistance in the liquid phase was neglected because of several effects that have been previously discussed. The air in the region near the surface is saturated with water, and evaporative factors on mass transfer have not been included in the analysis. The air temperature is assumed to be 25 C (77 F), and the atmospheric pressure is 101,325 Pascal (1 atmosphere). The physical properties of air are:

$$\begin{matrix}molecular weight of air = M = 28.9625 \\ density = rho = 1.18 kg / {m}^{3} ( 0.00118 g / {cm}^{3} ) \\ molar density = c = 0.04088 kmol / {m}^{3} \\ viscosity = mu = 2.0 \; 00 E - 5 kg / ( ms ) or 0.02 cP \\ molecular diffusivity for CO 2 in air = Dab = 1.6086 E - 5 m \hat{} 2 / s \\ molecular diffusivity for O 2 in air = Dab = 2.0748 E - 5 m \hat{} 2 / s\end{matrix}$$

Note the transport of oxygen (O2) will be more rapid than the heavier carbon dioxide (CO2). The following can be readily divided by the ratio 2.0748/1.6086=1.29, the ratio of the diffusivities, to obtain the carbon dioxide flux.

The Schmidt number, Sc, is calculated for oxygen and carbon dioxide as

$$Sc ( O 2 ) = mu / ( rho * Dab ) = [ 2 E - 5 k / ( ms ) ] / 1.18 kg / {m}^{3} * 2.0748 E - 5 m \hat{} 2 / s ] = 0.817$$

$$Sc ( CO 2 ) = mu / ( rho * Dab ) = [ 2 E - 5 k \text{g/} ( ms ) ] / 1.18 kg / {m}^{3} * 1.6086 E - 5 m \hat{} 2 / s ] = 1.05 .$$

The wind speed is assumed to be a constant value of 2 meters/second (or 4.47 miles per hour) and the mass transfer length, which is significantly less than the algal aquaculture pond length, is assumed to be one meter, so the Reynolds number is

$$R e = length * velocity * rho / mu = ( 0.5 m ) * ( 2 m / s ) * ( 1.18 kg / m 3 ) / ( 2 E - 5 kg / ( ms ) )$$

$$Re = 118 , 000$$

$$The mass flux , jD is 0.0019298 since$$

$$jD = 0.6 \; 64 R e \hat{} ( - 0.5 )$$

$$or$$

$$jD = ( Sh / ( R e * Sc ) ) * Sc \hat{} 2 / 3 = 0. 6 \; 64 R e \hat{} ( - 0.5 ) .$$

Thus, the Sherwood number, Sh, which is the rate of mass transfer relative to the diffusion rate, is 232 for carbon dioxide, and 213 for oxygen:

$$Sh ( C O 2 ) = 0.664 * {Re}^{⋀} ( 0 \text{.5} ) * Sh {(}^{⋀} 1 / 3 ) = 0.664 * 118 , 00 {0}^{⋀} 0.5 * {1.05}^{⋀} ( 1 / 3 ) = 232$$

$$Sh ( O 2 ) = 0.664 * {Re}^{⋀} ( 0 \text{.5} ) * Sh {(}^{⋀} 1 / 3 ) = 0.664 * 118 , 00 {0}^{⋀} 0.5 * {0.817}^{⋀} ( 1 / 3 ) = 213$$

The diffusional flux is computed from

$$Diffusional flux = c * Dab / length = ( 0.04088 kmol / m3 ) * ( 1.6086 E - 5 {m}^{⋀} 2 / s ) / ( 1 m )$$

$$Diffusional flux for carbon dioxide is = 6.57 E - 7 kmol / {m}^{2} / s$$

$$Diffusional flux = c * Dab / length = ( 0.04088 kmol / {m}^{3} ) * ( 2.07 \; 48 E - 5 {m}^{⋀} 2 / s ) / ( 1 m )$$

$$Diffusional flux for oxygen is = 8.48 E - 7 kmol / {m}^{2} / s$$

Since the Sherwood number is the mass transfer coefficient divided by the diffusional flux, the mass transfer coefficient (kyprime) is computed to be:
For carbon dioxide as:

$$kyprime = Sh * diffusional flux = ( 232 ) * ( 6 \text{.57} {E}^{- 7} kmol / {m}^{2} / s ) = 1.62 {E}^{- 5} kmol / {m}^{2} / s$$

$$kyprime = 0.55 kmol / {m}^{2} / hr$$

$$kyprime = 13.2 kmol / {m}^{2} / day$$

$$kyprime = 4815 kmol / {m}^{2} / yr$$

For oxygen as:

$$kyprime = Sh * diffusional flux = ( 213 ) * ( 8.48 {E}^{- 7} kmol / {m}^{2} / s ) = 1.81 {E}^{- 5} kmol / {m}^{2} / s$$

$$kyprime = 0.65 kmol / {m}^{2} / hr$$

$$kyprime = 15.6 kmol / {m}^{2} / day$$

$$kyprime = 5701 kmol / {m}^{2} / yr$$

The bulk O2 concentration is 20.9 wt %, and the interfacial O2 concentration is 21.0 wt %, the log mean oxygen driving force is 0.7905

$$Delta = 0.21 - 0.209 = 0.001$$

$$yAirLM = ( ( 1 - 0.21 ) - ( 1 - 0.209 ) ) / LN ( ( 1 - 0.21 ) / ( 1 - 0.209 ) ) = 0 \text{.7905}$$

$$The driving force is 0.001 / 0.7905 = 1.26 E - 3$$

The mass flux of oxygen then is the mass transfer coefficient multiplied by the driving force, or

$$Noxygen = ( 126 E - 3 ) * ( 5701 kmol / m 2 / yr ) = 7.21 kmol / m 2 / yr = 26.3 g of O 2 / {m}^{2} / hr$$

Thus, a tremendous amount of oxygen can be removed from an algal culture just based on diffusional mass transfer from an unagitated algal aquaculture pond. The production of oxygen in an algal aquaculture pond (the nutrient recovery facility) will be a function of the quantity of algal biomass, the temperature, and the amount of solar radiation. Each of these factors will vary from day to day, season to season. There is a light-dark bottle technique for measuring oxygen production to determine net primary productivity. At the peak of the solar radiation during the day the oxygen levels in an algal aquaculture pond might reach 150 to 200% of saturation. If saturation of oxygen is 8.7 mg/L then the oxygen levels can be as high as 17.4 mg/L during the peak hour of oxygen production. An estimate for oxygen production rates might be in the 8-12 mg/L/hr range. Rates have been measured in a photobioreactor of 4-6 mmoles/L/hr or 128 to 192 mg/L/hr. To take into effect the amount of algal biomass a specific oxygen production rate is calculated by dividing by this quantity to give units of mg O₂/mg biomass/hr. In this example, if the biomass concentration is 8,000 mg/L then the specific oxygen production rate would be 0.016-0.024 mg O₂/mg biomass/hr (0.016-0.024 hr⁻¹).

### Example 2: Carbon Dioxide Transport into a Brine Aquaculture System

Carbon dioxide is transported into an extensive algal aquaculture pond containing Dunaliella salina. The dominant resistance to carbon dioxide mass transfer into the algal aquaculture pond is assumed to be in the gas phase. Otherwise, the biology would set the mass transfer rates, instead of the diffusional mass transfer processes. The air temperature is 25° C. (77° F.), and the atmospheric pressure is 101,325 Pascal (1 atmosphere). The rate of carbon dioxide mass transfer into the algal aquaculture pond is calculated below to assure that the rate is higher than the annually assumed biomass productivity. The physical properties of air are:

$$molecular weight of air = M = 28.9625$$

$$density = rho = 1.18 kg / {m}^{3} ( 0.00118 g / {cm}^{3} )$$

$$molar density = c = 0.0 \; 4088 kmol / {m}^{3}$$

$$viscosity = mu = 2. E - 5 kg / ( ms ) or 0.02 cP$$

$$molecular diffusivity of CO 2 in air = Dab = 1.6086 E - 5 {m}^{⋀} 2 / s$$

The Schmidt number, Sc, is calculated as

$$Sc = mu / ( rho * Dab ) = [ 2 E - 5 kg / ( ms ) ] / [ 1.18 kg / m 3 * 1.6086 E - 5 {m}^{⋀} 2 / s ] = 1.05$$

The wind speed is a constant 2 meters/second, or 4.47 miles per hour, and the algal aquaculture pond length is assumed to be one meter, so the Reynolds number is

$$Re = length * velocity * rho / mu = ( 1 m ) * ( 2 m / s ) * ( 1.18 kg / m 3 ) / ( 2 E - 5 kg / ( ms ) )$$

$$Re = 118 , 000$$

The mass flux, jD is 0.0019298 since

$$jD = 0.6 \; 64 {Re}^{⋀} ( - 0 \text{.5} )$$

$$or$$

$$jD = ( Sh / ( Re * Sc ) ) * {Sc}^{⋀} 2 / 3 = 0.6 \; 64 {Re}^{⋀} ( - 0.5 )$$

Thus, the Sherwood number, Sh, which is the rate of mass transfer relative to the diffusion rate, is 232

$$Sh = 0.664 * {Re}^{⋀} ( 0 \text{.5} ) * Sh {(}^{⋀} 1 / 3 ) = 0.664 * 118 , 00 {0}^{⋀} 0.5 * {1.05}^{⋀} ( 1 / 3 ) = 232$$

The diffusional flux is computed from

$$Diffusional flux = c * Dab / length = ( 0.04088 kmol / m3 ) * ( 1.6086 E - 5 {m}^{⋀} 2 / s ) / ( 1 m )$$

$$Diffusional flux = 6.57 E - 7 kmol / m2 / s$$

Since the Sherwood number is the mass transfer coefficient divided by the diffusional flux, the mass transfer coefficient (kyprime) is computed to be:

$$kyprime = Sh *= ( 232 ) * ( 6.57 {E}^{- 7} kmol / {m}^{2} / s ) = 162 {E}^{- 5} kmol / {m}^{2} / s$$

$$kyprime = 0.55 kmol / {m}^{2} / hr$$

$$kyprime = 13.2 kmol / {m}^{2} / day kyprime = 4815 kmol / {m}^{2} / yr$$

The bulk CO2 concentration is conservatively assumed to be 365 ppm and the interfacial CO2 concentration is zero, as all of the CO2 is consumed as it enters the aquaculture medium. Thus, the log mean oxygen driving force, yCO2LM is:

$$Delta = 0.000365 - 0 = 0 \text{.000365}$$

$$yCO 2 LM = ( ( 1 - 0.000365 ) - ( 1 - 0 ) ) / LN ( ( 1 - 0.000365 ) / ( 1 - 0 ) ) = 0.9 \; 998$$

$$The driving force is 0. 0 \; 365 / 0.9998 = 3.65 {E}^{- 4}$$

The mass flux of carbon dioxide, Ncarbon dioxide, then is the mass transfer coefficient multiplied by the driving force, or

$$Ncarbon = ( 3.6 5 {E}^{- 4} ) * ( 4815 kmol / {m}^{2} / yr ) = 1.76 kmol C O 2 / {m}^{2} / yr = 77.4 kg C O 2 / {m}^{2} / yr = 212 g C O 2 / {m}^{2} / day = 57.8 g carbon / {m}^{2} / day .$$

Since algal biomass is about 50% by weight carbon, this carbon dioxide mass transfer rate provides enough transport to grow 115.6 grams of algal biomass/m²/day. This value is significantly more than the algal productivity of racetrack algal aquaculture ponds, with the reported aerial productivity of about 30 to 60 g biomass/m²/day. Therefore, the use of agitated algal aquaculture ponds, such as those agitated with paddle wheels, is not necessary to supply carbon dioxide to the algae. Furthermore, the need to bubble stack gas or carbon dioxide into an algal aquaculture pond is not necessary to provide rapid algal growth.

### Example 3. Wind Speed and Fetch Influence on the Wave Mixed Layer of an Algal Aquaculture Pond

Using the equations presented in Carper and Bachmann, the wave mixed layer (WML) can be computed for a given Fetch and wind speed. Likewise, the Fetch can be calculated to provide a certain WML for a given wind speed.

For a wind speed 5 of 1 m sec⁻¹ (2.2 mph) it takes a Fetch 3 of 470 m to provide a WML 7 of 0.15 m. At this Fetch a 2 m sec⁻¹ (4.5 mph) wind speed will generate a WML of 0.33 m. It is interesting to note that a wind speed of 2 m sec⁻¹ will not give a WML>1.85 m no matter how long the Fetch. At a Fetch of 470 m, a 2 m sec⁻¹ wind speed will give a WML of 0.33 m.

In the examples, the length of the algal growth algal aquaculture pond was 1000 m. Using this distance as the Fetch, a 1 and 2 m sec⁻¹ wind speed provides a WML of 0.19 m and 0.45 m, respectively. To generate a WML of 2 m at a Fetch of 1000 m it takes a 7.75 m sec⁻¹ (17.3 mph) wind speed.

To obtain a WML of 2 m, it takes combinations of Fetch and wind speed of:

| Fetch, m | Wind Speed, m sec⁻¹ (mph) |
|---|---|
| 500 | 10.69 (23.9) |
| 1000 | 7.75 (17.3) |
| 1500 | 6.47 (14.5) |
| 2000 | 5.72 (12.8) |
| 2500 | 5.22 (11.7) |
| 3000 | 4.86 (10.9) |
| 3500 | 4.58 (10.2) |

If the minimum slope of the algal aquaculture pond is 0.05 m for every 100 m, one might take another approach. If the inlet depth is 0.15 m, then the outlet depth can be determined and using wind speeds of 1 and 2 m sec⁻¹ the distance along the Fetch that is mixed to the bottom can be calculated.

| | | WML at a | Distance | WML at a | Distance |
|---|---|---|---|---|---|
| | Outlet depth, | wind speed of | mixed to | wind speed of | mixed to |
| Fetch, m | m | 1 m sec⁻¹ | bottom, m | 2 m sec⁻¹ | bottom, m |
| 500 | 0.40 | 0.15 | — | 0.34 | 380 |
| 1000 | 0.65 | 0.19 | 80 | 0.45 | 600 |
| 1500 | 0.90 | 0.22 | 140 | 0.53 | 760 |
| 2000 | 1.15 | 0.24 | 180 | 0.60 | 900 |
| 2500 | 1.40 | 0.26 | 220 | 0.65 | 1000 |
| 3000 | 1.65 | 0.27 | 240 | 0.69 | 1080 |
| 3500 | 1.90 | 0.28 | 260 | 0.73 | 1160 |

In one exemplary embodiment of the disclosure, the ratio of a wave mixed layer of the algal growth medium to a depth of the algal growth medium in the algal aquaculture pond is greater than about 0.2, such as greater than about 0.3, greater than about 0.4, greater than about 0.5, greater than about 0.6, greater than about 0.7, greater than about 0.8, greater than about 0.9 or greater than about 1.0.

### Example 4: Data for Examples E1-E9 Using Samples Collected at Seven Sites of a 400 Hectare Algal Aquaculture Pond

Samples were collected at seven different sites located around the perimeter of a 400 hectare algal aquaculture pond 1. The samples were collected by mixing the entire contents of the pond in a two meter semi-circle around the sample location. The number of D. salina algal cells per milliliter of solution were quantified for each sample. The carotenoid content in each sample was also quantified. The cell count was determined by placing a drop of the sample on a hemocytometer and counting the number of D. salina cells with a light microscope within hours of the sample collection time. The carotenoid content in each sample was quantified by intimately contacting a known volume of the sample with a known amount of chloroform within hours of sample collection, and determining the carotenoid concentration by UVIVIS spectroscopy measurement at 456 nanometers using a calibration curve. The mean and standard deviation for both the cell counts per milliliter and the carotenoid concentration were computed for the samples collected at the seven different sites. The pond was not lined with plastic or clay, but had an earthen bottom, and the borders were constructed of soil and rock. The shape of the pond was trapezoidal with the maximum and minimum Fetch of 2800 and 1250 meters, respectively. The mean pond depth was 0.5 meters, and the pond was nearly uniform in depth. The algal growth pond was operated with continuous flow both in and out of the pond. The nitrogen and phosphorus nutrient levels were maintained as a constant within the pond. No rainfall was recorded during the 24 hour preceding the collection of the samples, and there was no fresh water layer on the pond surface. A sample of the algal growth pond discharge (outlet sample) was collected at the exit weir so the sample was well mixed.

Both the cell count and the carotenoid content of the outlet sample were determined with the identical methods described above. The mean wind speed for the six hours prior to collecting the sample was measured at an airport weather station located about 30 km away from the location of the algal growth pond. The mean wind speed was 3.1 meters per second and the Fetch was 1660 meters. Note, the average wind speed for the month was 3.3 meters per second and the historical average wind speed from 1972 to 2021 was 3.2 meters per second (Data from the local airport and wind rose from https://mesonet.agron.iastate.edu/sites/locate.php). Based on these measurements, the Wave Mixed Layer (WML) was calculated to be 106 centimeters using the equation WML (cm)=0.83×Wind speed (m/s)×Fetch (meters). Defining the Wind Mixing Parameter as the dimensionless ratio of the wave mixed layer divided by the mean pond depth, the Wind Mixing Parameter (WMP) was 2.1 (greater than 0.2). The cell count and the carotenoid concentration of the outlet sample were both found to be within two standard deviations (a 95% confidence level) of the seven samples collected around the pond. Thus, the contents of the algal growth pond were well mixed with the WMP of 2.4 and behaved as a continuous-flow stirred tank (CSTR). The Reynolds number for this case was also computed where the velocity component was defined as the mean wind velocity and the length was defined as the mean pond depth. The density and viscosity of the brine were 1200 kg/m³ and 1.1 centipoise, respectively. The resulting Reynolds number was 1,706,880. The mean ratio of the carotenoid to chlorophyll content in the pond was controlled to be relatively constant based on a constant flow of algae, growth medium, and nutrients into and out of the pond.

Data for Examples E1 through E9 are given in Table 2 below. Note, carotenoid (bc) data were not collected for Examples E6 and E7, while cell count data were not collected for Examples E8 and E9.

TABLE 2
for Examples E1-E9.

| | Temp | | | Wind speed- | | Wind | Wave | | | Pond |
|---|---|---|---|---|---|---|---|---|---|---|
| | at | | | average | | speed | mixed | Reynolds | Pond | stand |
| Example | 1200 | Humidity | Conditions | 0600 to 1500 | Fetch | average | layer | number | average | dev |
| Number | (F.) | (%) | (obs) | (mph) | (m) | (m/s) | (cm) | (—) | (ppm bc) | (ppm bc) |
| E1 | 73° F. | 26% | Fair | 7.0 | 1660.0 | 3.1 | 106.8 | 1 706 880 | 0.18 | 0.11 |
| E2 | 76° F. | 27% | Mostly | 7.0 | 1250.0 | 3.1 | 91.8 | 1 706 880 | 0.24 | 0.08 |
| | | | Cloudy | | | | | | | |
| E3 | 85° F. | 19% | Partly | 7.0 | 1600.0 | 3.1 | 103.9 | 1 706 880 | 0.32 | 0.24 |
| | | | Cloudy | | | | | | | |
| E4 | 73° F. | 40% | Fair | 4.0 | 1660.0 | 1.8 | 60.5 | 975 360 | 1.29 | 0.76 |
| E5 | 82° F. | 29% | Fair | 7.0 | 1660.0 | 3.1 | 106.8 | 1 706 880 | 1.89 | 1.79 |
| E6 | 86° F. | 32% | Mostly | 12.0 | 1250.0 | 5.4 | 157.4 | 2 926 080 | NA | NA |
| | | | Cloudy | | | | | | | |
| E7 | 89° F. | 22% | Fair | 5.0 | 1660.0 | 2.2 | 75.6 | 1 219 200 | NA | NA |
| E8 | 89° F. | 17% | Partly | 8.0 | 1660.0 | 3.6 | 120.9 | 1 960 720 | 0.46 | 0.36 |
| | | | Cloudy | | | | | | | |
| E9 | 88° F. | 27% | Fair | 6.2 | 1600.0 | 2.8 | 92.0 | 1 511 808 | 0.79 | 0.62 |

| | Pond + | Pond − | Pond | | Pond | Pond + | Pond − | Pond |
|---|---|---|---|---|---|---|---|---|
| | 2 | 2 | outlet | Pond | stand | 2 | 2 | outlet |
| Example | SD | SD | flow | average | dev | SD | SD | flow |
| Number | (ppm bc) | (ppm bc) | (ppm bc) | (cells/ml) | (cells/ml) | (cells/ml) | (cells/ml) | (cells/ml) |
| E1 | 0.39 | −0.04 | 0.10 | 2214 | 1475 | 5164 | −736 | 3000 |
| E2 | 0.40 | 0.07 | 0.12 | 2107 | 1442 | 4992 | −778 | 2500 |
| E3 | 0.80 | −0.16 | 0.12 | 2821 | 2486 | 7794 | −2151 | 4000 |
| E4 | 2.80 | −0.23 | 1.64 | 14571 | 7959 | 30490 | −1347 | 8250 |
| E5 | 5.48 | −1.70 | 1.23 | 17443 | 14945 | 47334 | −12448 | 14000 |
| E6 | NA | NA | NA | 24296 | 7986 | 40258 | 8313 | 32000 |
| E7 | NA | NA | NA | 2680 | 2129 | 8919 | −1599 | 2000 |
| E8 | 1.18 | −0.26 | 0.43 | NA | NA | NA | NA | NA |
| E9 | 2.02 | −0.45 | 0.55 | NA | NA | NA | NA | NA |

Nomenclature for Table 2. SD=standard deviation. Note: Wind Rose Data: Wind rose data should be collected from the nearest airport to the facility and if there are multiple options, the one that is nearest to the coastline should be used.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment. That said, it is understood that any one or more features disclosed herein may be combined.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present disclosure may be referred to herein along with alternatives for the various components thereof. It is understood that such embodiments, examples, and alternatives are not to be construed as de facto equivalents of one another but are to be considered as separate and autonomous representations of the present disclosure.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the disclosure. One skilled in the relevant art will recognize, however, that the disclosure can be practiced without one or more of the specific details, or with other methods, components, materials, etc. In other instances, well-known structures, materials, or operations are not shown or described in detail to avoid obscuring aspects of the disclosure.

While the foregoing embodiments and examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the disclosure. Accordingly, it is not intended that the disclosure be limited, except as by the claims set forth below.

The following patent documents and literature are also incorporated by reference herein in their entirety.
U.S. Pat. No. 4,958,460 September 1990 Nelson et al.
U.S. Pat. No. 7,662,616
Astarita, G., D. W. Savage, and A. Bisio, Gas Treating with Chemical Solvents, Wiley, New York, NY, Chapter 9 (1983).
Bear, J Dynamics of Fluids in Porous Media. Dover Publications, p. 136 (1972).
Carper, G. L., and R. W. Bachmann, “Wind Resuspension of Sediments in a Prairie Lake,” Can. J. Fish. Aquat. Sci. 41:1763-1767 (1984).
Crowl, D. A. and J. F. Louvar, Chemical Process Safety, Fundamentals with Applications, 2^{nd} ed., Prentice Hall PTR, Englewood Cliffs, NJ (2002).
Cullinane, J. T. and G. T. Rochelle, “Kinetics of Carbon Dioxide Absorption into Aqueous Potassium Carbonate and Piperazine”, Ind. Eng. Chem. Res. 2006, 45 (8) pp. 2531-2545 (2006).
Dankwerts, P. V. Gas-Liquid Reactions, McGraw Hill, New York, NY (1970).
Kohl, A. L., Riesenfeld, F, Gas Purification, 3^{rd}.ed. Gulf Publishing, Houston (1979).
Putt, R. Algae as a Biodiesel Feedstock: A Feasibility Study, Appendix A: Air-to-Algal aquaculture pond Carbon Dioxide Transport, Report to NREL (2008).
Sherwood, T. K., R. L Pigford, and C. R. Wilke, Mass Transfer, McGraw Hill, New York, NY, Chapter 8 (1975).
Skelland, A. H. P., Diffusional Mass Transfer, Wiley, New York, NY (1974).
Treybal, R. E. Mass Transfer Operations, 3^{rd} ed. McGraw Hill Book Company, New York, NY, Chapter 3 (1980).
Wanninkhof, R., Journal of Geophysical Research, 97 C5, p. 7373-7382 (1992).
Guidelines for Chemical Process Quantitative Risk Analysis, 2^{nd} ed. Center for Chemical Process Safety, American Institute of Chemical Engineers, New York (2000).

1. A process of culturing algae and mixing algal growth medium in at least one open algal aquaculture pond for a continuous flow system, the process comprising:
(a) constructing the open algal aquaculture pond, the algal aquaculture pond possessing a fetch selected as a function of a specified wind speed and a wave mixed layer (WML) defined by a specified depth from the surface of the algal aquaculture pond;
(b) supplying algal growth medium to the open algal aquaculture pond, the open algal aquaculture pond having at least partially a manmade configuration and being in communication with a harvester for separating algal biomass from the algal growth medium; and
(c) culturing algae in the algal growth medium in the open algal aquaculture pond by mixing at least part of the algal growth medium and/or circulating the algal growth medium within the algal aquaculture pond, the mixing being a function of the wind speed and the fetch;
and wherein a ratio of the WML of the algal growth medium to a total depth of the algal growth medium in the algal aquaculture pond is greater than about 0.2.

2. The process according to claim 1, comprising:
the open algal aquaculture pond being configured to continuously remove the algal growth medium from the open algal aquaculture pond.

3. The process according to claim 1, wherein the WML is selected as a function of a constant, a specified wind speed, and a specified fetch.

4. The process according to claim 1, wherein the fetch is specified as an average distance wind will travel across the open algal aquaculture pond.

5. The process according to claim 1, wherein the fetch is the length of the algal aquaculture pond in a direction of wind travel.

6. The process according to claim 1, wherein the fetch is a distance selected to be at least one or more of about 75 to about 3000 meters, from about 80 to about 2000 meters, and/or from about 100 to about 1500 meters, optionally across the longest dimension of the algal aquaculture pond.

7. The process according to claim 1, wherein the ratio of the WML of the algal growth medium to the total depth of the algal growth medium in the algal aquaculture pond is selected to be at least one or more of greater than about 0.3, about 0.4, about 0.5, about 0.6, about 0.7, about 0.8, about 0.9, and/or about 1.0.

8. The process according to claim 1, wherein the algal growth medium comprises algal nutrients such as added algal nutrients.

9. The process according to claim 1, wherein the total depth of the open algal aquaculture pond is selected to be at least one or more of variant and/or consistent.

10. The process according to claim 1, wherein the total depth of the algal aquaculture pond aids the WML to promote mixing, and/or the total depth at any relative position is from about 0.15 m to about 4 m.

11. The process according to claim 1, wherein the open algal aquaculture pond contains an average algal cell concentration of about 20,000 to about 2,000,000 algal cells per mL.

12. The process according to claim 1, wherein the open algal aquaculture pond is an open algal aquaculture pond of a selected volumetric capacity for optimal mixing.

13. The process according to claim 1, wherein the open algal aquaculture pond includes at least one dimension larger than 50 m, and/or a mean depth of less than 4 m.

14. The process according to claim 1, wherein the open algal aquaculture pond has an algal aquaculture pond size selected to be at least one or more of greater than about 10, about 20, about 50, about 100 hectares, and/or up to about 1000 hectares; optionally wherein the size of the algal aquaculture pond is from about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, about 5-about 10 hectares, or about 100-about 1000 hectares.

15. The process according to claim 1, comprising:
utilizing mechanical means for mixing, or not including a step of utilizing mechanical means for mixing.

16. The process according to claim 1, wherein the open algal aquaculture pond is built on a foundation with permeability for holding algal growth medium containing algae whilst facilitating the wind (or the WML).

17. The process according to claim 16, wherein the foundation includes clay, rock, or concrete, or a combination thereof.

18. The process according to claim 1, wherein the open algal aquaculture pond has a non-uniform cross-section.

19. The process according to claim 1, wherein the open algal aquaculture pond has a configuration that is selected to be at least one or more of circular, oval, square, triangular, trapezoidal, and/or rectangular, or any combination thereof.

20. The process according to claim 1, wherein the WML is obtained using WML=0.830 (wind speed) (√fetch), wherein 0.830 is the constant.

21. The process according to claim 1, wherein one or more algal nutrients are applied to the algal growth medium for optimal algal growth.

22. The process according to claim 21, wherein the one or more algal nutrients are applied in the form selected to be at least one or more of a liquid solution, as a slurry, as a solid, and/or as machine processed pellets, or any combination thereof.

23. The process according to claim 1, wherein the WML is measured by e^{[-0.18691+0.48936*In (fetch)+1.0365*In (wind speed)]}.

24. The process according to claim 1, wherein the open algal aquaculture pond contains a constant carotene/chlorophyll ratio.

25. The process according to claim 1, wherein the open algal aquaculture pond comprises algae selected to be at least one or more of: Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia losterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and/or genetically-engineered varieties, or any combination thereof.

26. The process according to claim 1, wherein the algal growth medium contains a salinity from about 5 wt % to about 27 wt %.

27. A continuous flow algal culturing system for mixing an algal growth medium, the system comprising:
at least one or more of an open algal aquaculture pond, the open algal aquaculture pond and possessing a fetch selected as a function of a specified wind speed and a wave mixed layer (WML) defined by a specified depth from the surface of the algal aquaculture pond;
at least a part of the open algal aquaculture pond having at least partially a manmade configuration and being in communication with a harvester for separating algal biomass from the algal growth medium; and
wherein a ratio of the WML of the algal growth medium to a total depth of the algal growth medium in the algal aquaculture pond is greater than about 0.2.

28. The system according to claim 27, comprising:
at least one device to a) monitor, supply and/or maintain target nutrient concentrations for optimal algal growth, and/or b) monitor, supply and/or maintain average concentrations of the algae and the nutrients at greater than or equal to their concentrations in an effluent flow.

29. The system according to claim 27, consisting of a plurality of interconnected algal aquaculture ponds containing selected fetches.

30. The system according to claim 27, wherein the open algal aquaculture pond is connected to at least one algal pre-treatment unit and/or at least one extraction unit.

31. The system according to claim 27, wherein the harvester includes at least one or more of: an adsorptive bubble separation unit, a centrifugation unit, a flocculation unit, a sedimentation unit, and/or a filtration unit, or any combination thereof.

32. A mixed algal aquaculture medium obtainable by a process according to claim 1.

33. Use of a mixed algal aquaculture medium according to claim 32 or a system.
